(19) United States
(12) Patent Application Publication
(10) Pub. No.: US 2015/0079132 A1
(43) Pub. Date: Mar. 19, 2015

(54) EVOKING PROTECTION AGAINST STREPTOTOCCUS PNEUMONIAE INCORPORATING B-CELL AND T-CELL PNEUMOCOCCAL PROTEIN ANTIGENS AND PNEUMOCOCCAL POLYSACCHARIDES DELIVERED CONCOMITANTLY

(71) Applicant: PATH, Seattle, WA (US)

(72) Inventors: Jean-Francois Lucien Maisonneuve, Federal Way, WA (US); Mark Raymond Alderson, Bainbridge Island, WA (US)

(73) Assignee: PATH, Seattle, WA (US)

(21) Appl. No.: 14/489,104

(22) Filed: Sep. 17, 2014

Related U.S. Application Data
(60) Provisional application No. 61/879,040, filed on Sep. 17, 2013.

(51) Int. Cl. A61K 39/09

(52) U.S. Cl. CPC A61K 39/092
USPC 424/244.1

(57) ABSTRACT

This disclosure is directed to compositions that combine the polysaccharide-specific antibody protection afforded by the conventional vaccines through carrier effects provided by one or more pneumococcal common T-cell antigen(s) together with Streptococcus pneumoniae-specific Th-17 responses elicited by the pneumococcal carrier common T-cell antigen. The disclosed compositions are useful for pan-serotypic protection against NP carriage, and antibody responses against common pneumococcal virulence factors, potentially useful for pan-serotype protection against Streptococcus pneumoniae invasive diseases.

### CROSS-REFERENCE TO RELATED APPLICATION

This application claims the benefit of Provisional Application No. 61/879,040, filed Sep. 17, 2013, which is expressly incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 52641_Sequence_final_{—}2014-09-16.txt. The text file is 288 KB, was created on Sep. 16, 2014, and is being submitted via EFS-Web with the filing of the specification.

### TECHNICAL FIELD

The present disclosure generally relates to immunogenic compositions and vaccine formulations and their methods of use for stimulating a host response against Streptococcus pneumoniae antigens.

### BACKGROUND

Pneumococcal disease continues to be a leading cause of sickness and death in the United States and throughout the world. Each year, millions of cases of pneumonia, meningitis, bacteremia, and otitis media are attributed to infection with the pathogen Streptococcus pneumoniae. S. pneumoniae is a Gram-positive encapsulated coccus that colonizes the nasopharynx in about 5-10% of healthy adults and 20-40% of healthy children. Normal colonization becomes infectious when S. pneumoniae is carried into the Eustachian tubes, nasal sinuses, lungs, bloodstream, meninges, joint spaces, bones and peritoneal cavity. S. pneumoniae has several virulence factors that enable the organism to evade the immune system. Examples include a polysaccharide capsule that reduces phagocytosis by host immune cells, proteases that inhibit complement-mediated opsonization, and proteins that cause lysis of host cells. In the polysaccharide capsule, the presence of complex polysaccharides forms the basis for dividing pneumococci into different serotypes. To date, more than 90 serotypes of S. pneumoniae have been identified.

Various pharmaceutical compositions have been used to harness an immune response against infection by S. pneumoniae. A polyvalent pneumococcal vaccine, PPV-23, was developed for preventing pneumonia and other invasive diseases due to S. pneumoniae in the adult and aging populations. The vaccine contains capsular polysaccharides (CPs) from 23 serotypes of S. pneumoniae. As T cell-independent antigens, these CPs induce only short-lived antibody responses without immunological memory, thus necessitating repeated doses, which increases the risk of immunological tolerance. The antibodies raised against S. pneumoniae, termed anticapsular antibodies, are recognized as generally protective in adult and immunocompetent individuals. Also, carriage of pneumococci, wherein the bacteria are colonized in a subject's nasopharynx without causing symptoms of an active infection but are capable of being transferred to others, is not affected. Accordingly, this vaccine strategy does not promote indirect, or “herd” immunity. Furthermore, children under 2 years of age and immunocompromised individuals, including the elderly, do not respond well to T cell-independent antigens and, therefore, are not afforded optimal protection by PPV-23.

PREVNAR®, another S. pneumoniae vaccine, includes bacterial polysaccharides from seven S. pneumoniae strains conjugated to the mutated diphtheria toxin protein CRM₁₉₇. This vaccine induces both B and T cell immune responses. However, because it only protects against 7 pneumococcal serotypes, serotype replacement can render PREVNAR® less effective. Serotype emergence or replacement has already been demonstrated in several clinical trials and epidemiologic studies, necessitating development of different formulations of these vaccines. An example is the recently introduced PREVNAR 13®, directed to 13 pneumococcal serotypes. Furthermore, the two PREVNAR® conjugated formulations are expensive to manufacture, greatly limiting their availability in the developing world. PPV-23, which consists of 23 purified, but nonconjugated polysaccharides, has broader coverage, but does not provide protection to children under the age of 2 years, a population which is at the highest risk for pneumococcal disease.

Accordingly, S. pneumoniae remains a major health concern, especially in very young, elderly, or immunocompromised patients. DNA and protein sequence information for S. pneumoniae has been known for some time facilitating research into alternative antigens or vaccine strategies. However, a major problem remains regarding how to elicit an immune response that is long-lived, is effective in all age groups, and is effective across a large spectrum of serotypes.

Thus, there remains a need to design more effective pharmaceutical compositions and methods than the current strategies offer. In particular, such compositions and methods need to incorporate combinations of antigens that elicit a balanced and enduring immune response against multiple S. pneumoniae serotypes in an effort to ameliorate symptoms of septic infections and to reduce carriage. The present disclosure provides an approach addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides an immunogenic composition, vaccine formulation, and/or method of use that incorporates a novel combination of B cell and T cell antigens. The composition comprises i) a capsular polysaccharide (CP) component capable of stimulating B cell responses, and ii) a polypeptide component with at least a first polypeptide antigen and a second polypeptide antigen. The first polypeptide antigen is capable of inducing a T cell response, while the second polypeptide antigen is capable of inducing a B cell response. At least a portion of the CP component is conjugated to at least a portion of the polypeptide component that includes the first polypeptide antigen.

In some embodiments, the CP component comprises a plurality CP antigens derived from different S. pneumoniae serotypes. In some embodiments, the plurality of different S. pneumoniae CPs are derived from a plurality of S. pneumoniae serotypes comprising serotypes 1, 2, 3, 4, 5, 6B, 7F, 8, 9N, 9V, 10A, 11A, 12F, 14, 15B, 17F, 18C, 19F, 19A, 20, 22F, 23F, and/or 33F. In some embodiments, the different S. pneumoniae CPs are derived from a plurality of S. pneumoniae comprising serotypes 1, 4, 5, 6A, 6B, 7F, 9V, 14, 18C, 19A, 19F, and/or 23F.

In some embodiments, the first polypeptide antigen is capable of inducing a T_{H}17 cell response. In certain embodiments, the first polypeptide antigen has an amino acid sequence at least 90% identical to the polypeptide sequence of an antigen listed in Table 2, or any immunogenic fragment thereof. In some embodiments, the first polypeptide antigen has an amino acid sequence with at least 90% identity to SEQ ID NO:22 or SEQ ID NO:36, or any immunogenic fragment thereof.

In some embodiments, the second polypeptide antigen has an amino acid sequence at least 90% identical to the polypeptide sequence of an antigen listed in Table 1, or any immunogenic fragment thereof. In some embodiments, the second polypeptide antigen has an amino acid sequence with at least 90% identity to SEQ ID NO:90, or an antigenic fragment thereof, and comprises an L(Leu)460D(Asp) substitution.

In some embodiments, the first and second polypeptide antigens are linked. In certain embodiments, the first and second polypeptide antigens are domains of the same fusion polypeptide molecule. Thus, in further embodiments, at least a portion of the CPs is conjugated to the second polypeptide antigen.

In some additional embodiments, the first and second polypeptide antigens are separate polypeptide molecules.

In some embodiments, the immunogenic composition further comprises an adjuvant. In some embodiments, the adjuvant is an aluminum or aluminum salt-based adjuvant.

In some embodiments, the immunogenic composition is incorporated into a particle formulation.

In some embodiments, the administration of a therapeutically effective amount of the immunogenic composition to a subject in need results in reduced mucosal carriage of S. pneumoniae in the subject.

In some embodiments, the subject is a mammal, such as a mouse or human.

### DETAILED DESCRIPTION

The present disclosure provides immunogenic formulations and methods for generating an effective response against a broad spectrum of S. pneumoniae serotypes by targeting a plurality of target immune cell types.

Existing conjugated polysaccharide vaccines prevent many invasive pneumococcal diseases caused by vaccine-type strains. However, increased rates of disease caused by serotypes not covered by current vaccines have made creating a vaccine incorporating conserved pneumococcal protein antigens a priority. A vaccine based on noncapsular protein antigens that are well-conserved amongst the greater than 90 known pneumococcal serotypes would prevent immunologic escape through serotype replacement.

Expanded availability of pneumococcal genomic information has facilitated development of genome-based approaches for protein antigen identification. Efforts thus far have focused on identifying surface-exposed proteins that can be bound by circulating antibodies and thereby direct clearance of the pathogen through mechanisms similar to polysaccharide-based vaccines. However, it is currently unknown whether antibodies elicited against pneumococcal protein antigens will be as effective as anticapsular antibodies in providing protective immunity against targeted pneumococcus serotypes in humans.

During childhood, the incidence of pneumococcal disease caused by a broad range of serotypes declines years before natural acquisition of anticapsular antibodies suggesting other mechanisms provide natural immunity to pneumococcus. Studies in mice have shown that acquired immunity to pneumococcal colonization, either after mucosal exposure to live bacteria or elicited by intranasal immunization with killed unencapsulated pneumococcal whole-cell antigen (WCA), is antibody independent and CD4⁺ T cell dependent. This immunity was unchanged in mice that genetically lacked antibodies, IFNγ, or IL-4, but was completely abrogated in mice treated with neutralizing CD4 or IL-17A antibody or in mice genetically lacking the IL-17A receptor. This identifies the likely effector cells as IL-17A-producing CD4⁺ T_{H}17 cells. A similar role for IL-17 signaling in pathogen clearance has been observed in mouse models of infection for at least 12 other mucosal pathogens, indicating this pathway plays a general role in clearance of pathogens at mucosal surfaces. Furthermore, humans lacking T_{H}17 cells because of genetic mutation are highly susceptible to mucosal infections by pathogens such as Staphylococcus aureus, Haemophilus influenzae and S. pneumoniae (Milner et al., 2008, Nature, 452:773-776), indicating that T_{H}17 likely play an important role in natural immunity to important mucosal pathogens of humans.

Accordingly, the present disclosure provides for a multi-component vaccine, immunogenic compounds, and methods of use, that incorporate a polypeptide antigen component and a polysaccharide component. The polypeptide component comprises one or more polypeptide antigens that induce T_{H}17 cells. In some embodiments, these polypeptide antigens are useful to obtain responses against a broad spectrum of S. pneumoniae serotypes because they are not limited to particular serotypes defined by specific capsular polysaccharides. A T_{H}17-specific polypeptide component provides the additional benefit of enhanced protection against mucosal colonization and/or infection. This protection complements the enhanced protection provided by the traditional polysaccharide antigens and/or recognized surface-exposed polypeptide antigens that induce neutralizing antibody responses that can provide protection against invasive pneumococcal disease in an individual. Furthermore, combating the colonization of S. pneumoniae at mucosal surfaces, such as in the nasopharynx, contributes to protection against (or reduction of) asymptomatic carriage of pan-serotypic S. pneumoniae. A reduction in nasopharyngeal carriage, which may be related to IL-17 induction, provides an advantage at the host population level because of reduced transmission rates from vaccinated individuals, who may have otherwise been a carrier/transmitter. Thus, the vaccine formulations and associated methods that target, in part, a T_{H}17 cell response, can provide benefits beyond the subject receiving administration thereof to other members of the community. Accordingly in certain embodiments, the vaccine composition or immunogenic composition induces a T_{H}17 cell response greater than that induced by a control unrelated antigen (for example, the HSV-2 protein ICP47 with the gene name US12) after contacting T_{H}17 cells.

In some embodiments, the vaccine composition or immunogenic composition also induces a B-cell response that results in the production of antibodies specific for an S. pneumoniae antigen. In some embodiments, the polypeptide component comprises one or more polypeptide antigens that induce a B-cell response. In certain embodiments the polysaccharide component comprises one or more capsular polysaccharides (CPs) from S. pneumoniae serotypes that induce a B-cell response. In some embodiments, the one or more CPs are unconjugated. In some embodiments, the CPs are conjugated to a protein carrier. In some embodiments, the protein carrier induces a T-cell response, for example, a T_{H}17 cell response. The protein carrier can be any polypeptide described herein as part of the polypeptide component.

In a preferred embodiment, the vaccine composition or immunogenic composition induces a coordinate or concurrent increase in both a T_{H}17 cell response and a B-cell response. In some embodiments, the vaccine formulation inhibits infection by S. pneumoniae in an uninfected subject. In certain embodiments, the vaccine formulation reduces occurrence, duration or severity of S. pneumoniae nasopharyngeal colonization in an individual infected by S. pneumoniae. In some embodiments, the vaccine formulation inhibits development of sepsis in an individual infected by S. pneumoniae. In some embodiments, the vaccine formulation inhibits development of invasive diseases such as pneumonia, meningitis, otitis media, sinusitis or infection of other sites or organs with S. pneumoniae.

### A. Polysaccharide Antigens

The present embodiments incorporate one or more pneumococcal capsular polysaccharide (CPs) antigens into the vaccines and/or immunogenic compositions, and methods disclosed herein. As described above, CPs-based vaccines have been widely used to promote protective immunity against various specific serotypes of S. pneumoniae. Protective immunity is mainly dependent on the specific CPs used and the subject's production of anticapsular antibodies specific for the CP antigens. An unconjugated polyvalent vaccine, PPV-23 (PNEUMOVAX® 23, Merck Sharp & Dohme Corp., Whitestation, N.J.) incorporates CP antigens from S. pneumoniae serotypes 1, 2, 3, 4, 5, 6B, 7F, 8, 9N, 9V, 10A, 11A, 12F, 14, 15B, 17F, 18C, 19F, 19A, 20, 22F, 23F, and 33F. The combination of these CPs has been estimated to cover (i.e., induce antibodies in healthy individuals against) approximately 90% of the serious pneumococcal invasive disease serotypes in Western industrialized countries. However, it will be understood that CPs obtained from any S. pneumoniae serotype isolate are encompassed by the present disclosure.

Many of these polysaccharides are obtainable from the ATCC as lyophilized bulk powders. One preferred form of these polysaccharides is disclosed in U.S. Pat. No. 5,847,112, incorporated herein by reference. Alternatively, CPs can be isolated from bacterial cultures of S. pneumoniae. In this regard, the approach to isolation of the polysaccharides depends somewhat on the physical characteristics of the given CP. However, in general, the bacteria are cultured and the CPs are recovered according to known methods (see, e.g., Williams, C. A., and Chase, M. W., Methods in Immunology and Immunochemistry, Vol. I, Academic Press (1967)). In one embodiment, each pneumococcal serotype is grown in a soy-based medium. The individual CPs are then purified through standard steps including centrifugation, precipitation, and ultra-filtration. See, e.g., U.S. Pat. Pub. No. 2008/0286838 and U.S. Pat. No. 5,847,112, each of which are incorporated herein by reference.

Following a large scale culture of the bacteria in appropriate nutrient media known in the art to support Pneumococcal growth, a bactericidal, such as phenol or toluene, is added to kill the organisms. Alcohol fractionation of the polysaccharide is then conducted in two stages: a low alcohol stage to precipitate cellular debris and other unwanted impurities, and a water-miscible-alcohol stage to precipitate the capsular polysaccharides while leaving additional impurities in the supernatant fluid. Resuspension in an aqueous medium is followed by removal of contaminating proteins and nucleic acids by known methods such as nuclease or proteolytic digestion and/or solvent extraction. The crude polysaccharide is recovered by alcohol precipitation and drying to form a powder of the crude CPs. See, e.g, Example 3 of U.S. Pat. No. 5,623,057, incorporated herein by reference. These preparations can be useful, for example, for the inclusion of unconjugated CP antigens into the vaccines and immunogenic compounds of the present disclosure.

Capsular polysaccharides found to be poorly immunogenic by themselves have been shown to have improved immunogenicity when conjugated to an immunogenic carrier protein. Additionally, unconjugated CPs are poor inducers of T-cell immune responses. Accordingly, in some embodiments of the present disclosure, the CPs can be conjugated to a carrier protein or multiple carrier proteins. Carrier proteins are preferably proteins that are non-toxic and obtainable in a sufficient amount and purity. In some embodiments, the carrier proteins are themselves antigens, B-cell antigens or antigens capable of eliciting a T_{H}17 cell response. In some embodiments, the carrier protein is an antigen capable of eliciting a T_{H}17 cell response

Nonlimiting examples of carrier proteins for CP conjugation include DT (Diphtheria toxoid), TT (tetanus toxoid) or fragment C of TT, pertussis toxoid, cholera toxoid, E. coli LT, endotoxin A from Pseudomonas aeruginosa, and diptheria CRM₁₉₇. Bacterial outer membrane proteins can also be used, such as outer membrane complex c (OMPC) (e.g., outer membrane complex (OMPC) of Neisseria meningitides B), porins, transferrin binding proteins, pneumococcal surface protein A (PspA; see WO 2002/091998), pneumococcal adhesin protein (PsaA), C5a peptidase from Group A or Group B streptococcus, or Haemophilus influenzae protein D, pneumococcal pneumolysin (Kuo et al., 1995, Infect. Immun. 63:2706-2713) including pneumolysoid L460D (see, e.g., US 2009/0285846), pneumolysin (ply) detoxified in some fashion, for example dPLY-GMBS (see WO 2004/081515) or dPLY-formol, PhtX, including PhtA, PhtB, PhtD, PhtE and fusions of Pht proteins, for example PhtDE fusions, PhtBE fusions (see WO 2001/98334 and WO 2003/54007). Other proteins useful as carrier proteins for CP conjugate compositions include ovalbumin, keyhole limpet hemocyanin (KLH), bovine serum albumin (BSA) or purified protein derivative of tuberculin (PPD), PorB (from N. meningitidis), PD) (H. influenzae protein D; see, e.g., EP0594610B), or immunologically functional equivalents thereof, synthetic peptides (see EP0378881B and EP0427347B), heat shock proteins (see WO 93/17712 and WO 94/03208), pertussis proteins (see WO 1998/58668 and EP0471177B), cytokines, lymphokines, growth factors or hormones (see WO 1991/01146), artificial proteins comprising multiple human CD4⁺ T cell epitopes from various pathogen derived antigens (See Falugi et al., 2001, Eur. J. Immunol. 31:3816-3824) such as N19 protein (see Baraldoi et al., 2004, Infect. Immun. 72:4884-4887), iron uptake proteins (see WO 2001/172337), toxin A or B of C. dfficile (see WO 2000/61761), and flagellin (see Ben-Yedidia et al., 1998, Immunol. Lett. 64:9). CRM₁₉₇, for example, is a non-toxic variant (i.e., toxoid) of diphtheria toxin. In one embodiment, CRM₁₉₇ is isolated from cultures of Corynebacterium diphtheria strain C7 (β 197) grown in casamino acids and yeast extract-based medium.

Well-known examples of CP-conjugate vaccine compositions include PREVNAR® (Wyeth LLC/Pfizer, NY, N.Y.), a heptavalent CP conjugate vaccine that includes CPs from S. pneumoniae strains 4, 6B, 9V, 14, 18C, 19F, and 23F conjugated to CRM₁₉₇ of Corynebacterium diphtheriae; PREVNAR 13® (Wyeth LLC/Pfizer, NY, N.Y.) with the seven CPs of PREVNAR® plus six additional CPs from S. pneumoniae strains 1, 3, 5, 6A, 19A and 7F; and SYNFLORIX™ (GSK, Brentford, UK) a vaccine with the seven CPs of PREVNAR® plus CPs from S. pneumoniae strains 1, 5, and 7F, with eight of the CPs conjugated to a protein carrier from Haemophilus influenzae.

Vaccines and immunogenic compositions that incorporate multivalent conjugated CPs can include those with mixtures of different CP-protein conjugates, each conjugate prepared separately with a given CP subtype (i.e., from different serotypes). Alternatively or in addition, multivalent vaccines can include conjugates wherein several different CP subtypes are all conjugated to a given protein at one time or sequentially.

Crude CP extracts are often highly viscous and poorly soluble resulting in conjugates that are largely insoluble and unfilterable. Furthermore, the conjugation process from crude extracts results in poor yield and removal of unconjugated CPs, which is important for dosing reasons, is difficult for full length CPs. Thus, additional processing of prepared CPs can promote more efficient and effective incorporation into CP-protein conjugates. U.S. Pat. No. 5,623,057, incorporated herein by reference, provides an exemplary, non-limiting approach to preparing S. pneumoniae CPs useful for inclusion in protein-conjugate vaccines and immunogenic compositions. Disclosed herein are processing steps that can be employed in addition to the crude extract preparations described above that can facilitate and optimize the conjugation of CPs for vaccine formulations. Briefly, the dry, crude, capsular polysaccharide as prepared above can be purified, for example, by anion-exchange chromatography or other chromatographic procedure, prior to, or after partial hydrolysis. The chromatographic adsorption-desorption can be used either positively or negatively. Regardless of any purification steps, the CPs can be directly subjected to partial thermal hydrolysis, high-energy sonic hydrolysis, or other hydrolytic means, such as chemical, enzymatic or physical (e.g., a high pressure cell) means, which are known. A target endpoint of hydrolysis, conveniently measured by solution viscosity or high-performance size exclusion chromatography, is predetermined for each polysaccharide on a pilot scale such that antigenicity of the polysaccharide is not abrogated. For CPs that require more complex structure to retain antigenicity, a more gentle size reduction is achievable by sonic or physical shear means. Finally, the hydrolyzed CPs can be fractionated according to size and purity. Fractionation can be accomplished using differential alcohol solubility or chromatography using a size exclusion resin.

Many different schemes are available to those skilled in the art for preparing conjugates of polysaccharides and other moieties. Generally, the CPs are chemically activated to make the saccharides capable of reacting with the carrier protein. Once activated, each CP is separately conjugated to a carrier protein to form a glycoconjugate.

In one embodiment, the chemical activation of the polysaccharides and subsequent conjugation to the carrier protein are achieved by means described in U.S. Pat. No. 4,365,170, U.S. Pat. No. 4,673,574 and U.S. Pat. No. 4,902,506, each of which is incorporated herein by reference. Briefly, the chemistry entails the activation of pneumococcal polysaccharide by reaction with any oxidizing agent which oxidizes a terminal hydroxyl group to an aldehyde, such as periodate (including sodium periodate, potassium periodate, or periodic acid). The reaction leads to a random oxidative cleavage of vicinal hydroxyl groups of the carbohydrates with the formation of reactive aldehyde groups.

Coupling to the protein carrier (e.g., CRM₁₉₇) can be by reductive amination via direct amination to the lysyl groups of the protein. For example, conjugation is carried out by reacting a mixture of the activated polysaccharide and carrier protein with a reducing agent such as sodium cyanoborohydride. Unreacted aldehydes are then capped with the addition of a strong reducing agent, such as sodium borohydride.

In another embodiment, the conjugation method can rely on activation of the saccharide with 1-cyano-4-dimethylamino pyridinium tetrafluoroborate (CDAP) to form a cyanate ester. The activated saccharide can thus be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide, e.g., ethyl iodoacetimide HCl or N-succinimidyl bromoacetate or SIAB, or SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described in, for example, WO 1993/15760, WO 1995/08348, WO 1996/29094, Chu et al., 1983, Infect. Immunity 40:245-256, and the like.

Other suitable techniques use carbodiimides, hydrazides, active esters, norborane, p-nitrobenzoic acid, N-hydroxysuccinimide, S—NHS, EDC, TSTU. Many conjugation agents are described in WO 1998/42721, incorporated herein by reference in its entirety. Conjugation can involve a carbonyl linker which can be formed by reaction of a free hydroxyl group of the saccharide with 1,1′-carbonyldiimidazole (CDI) (see Bethell et al., 1979, J. Biol. Chem. 254:2572-2574; Hearn et al., 1981, J. Chromatogr. 218:509-518) followed by reaction of with a protein to form a carbamate linkage. This can involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

After conjugation of the capsular polysaccharide to the carrier protein, the polysaccharide-protein conjugates are purified (enriched with respect to the amount of polysaccharide-protein conjugate) by one or more of a variety of techniques. Examples of these techniques are well known to the skilled artisan and include concentration/diafiltration operations, ultrafiltration, precipitation/elution, column chromatography, and depth filtration. See, e.g., U.S. Pat. No. 6,146,902, incorporated herein by reference.

### B. Polypeptide Antigens

1. B-Cell Antigens

In some embodiments the vaccine or immunogenic composition comprises at least one S. pneumoniae antigen that is predominantly an antibody target. Exemplary antibody protein antigens are shown in Table 1. In some embodiments, this antigen is Pneumococcal surface adhesin A (PsaA) (SEQ ID NO:1) or fragments or variants thereof. In some embodiments, fragments or variants of PspA comprise proline-rich segments with the non-proline block (PR+NPB), for example the CD2 sequence (SEQ ID NO:2). In some embodiments, fragments or variants of PspA comprise proline-rich segments with the non-proline block and 10, 20 30, 40 or more additional amino acids of PspA sequence, for example the H70 sequence (SEQ ID NO:7). In some embodiments, the S. pneumoniae antigen that is predominantly an antibody target comprises a pneumolysoid. In some embodiments, the pneumolysoid is L460 pneumolysoid. These antibody target antigens are described in more detail below.

TABLE 1
Exemplary S. pneumoniae protein antigens are
predominantly antibody targets

| Locus tag name and description | Protein SEQ ID No. |
|---|---|
| PspA | 1 |
| PR + NRB from PspA with coiled-coil | 2 |
| CD2 | 3 |
| PR + NRB from PspA w/o coiled-coil | 4 |
| PR only with coiled-coil | 5 |
| PR only w/o coiled-coil | 6 |
| H70 (PR + NRB from PspA aa 290-410) | 7 |
| Non-proline Block (NPB) | 8 |
| Non-proline Block (NPB) | 9 |
| Non-proline Block (NPB) | 10 |
| S. pneumoniae protein pneumolysin | 90 |

In some embodiments, vaccines or pharmaceutical compositions comprising an S. pneumoniae polypeptide includes a fusion protein containing at least one S. pneumoniae antigen that is a B-Cell/antibody antigen. In some instances, the known S. pneumoniae antigens are predominantly antibody targets. In some instances, the known S. pneumoniae antigens protect from S. pneumoniae colonization, or from S. pneumonia-induced sepsis. One appropriate art-recognized class of S. pneumoniae antigen is Pneumococcal surface protein A (PspA) (SEQ ID NO:1) and derivatives of PspA. Derivatives of PspA include proline-rich segments with the non-proline block (PR+NPB, further described below as well as in Daniels, C. C. et al. (2010) Infect. Immun. 78:2163-72) and related constructs comprising all or a fragment of the proline-rich region of PspA (e.g., regions containing one or more of the sequences PAPAP (SEQ ID NO:91), PKP, PKEPEQ (SEQ ID NO:92) and PEKP and optionally including a non-proline block). H70 (SEQ ID NO:7) is one exemplary sequence which includes the proline-rich region and non-proline-block encompassing amino acids 290-410 PspA. An example of the non-proline-block has the exemplary sequence EKSADQQAEEDIYARRSEEEYNRLTQQQ (SEQ ID NO:8), which generally has no proline residues in an otherwise proline-rich area of the non-coiled region of PspA. Other embodiments of non-proline block (NPB) sequences include SEQ ID NOS:8 and 9 and PspA and its derivatives can include genes expressing similar proline-rich structures (i.e., PKP, PKEPEQ (SEQ ID NO:92) and PEKP), with or without the NPB. The amino acids at ether end of the NPB mark the boundaries of the proline-rich region. In one example, the amino-terminal boundary to the PR-region is DLKKAVNE (SEQ ID NO:11), and the carboxyterminal boundary is (K/G)TGW(K/G)QENGMW (SEQ ID NO:12). Peptides containing the NPB are particularly immunogenic, suggesting that the NPB can be an important epitope. Exemplary immunogenic PspA polypeptide derivatives containing the coiled-coil structure include SEQ ID NOS:2 and 5. Particular embodiments of the immunogenic PspA polypeptide derivatives lacking the coiled-coil structure have the amino acid sequences shown as SEQ ID NOS:3, 4 and 6. Immunogenic PspA polypeptides SEQ ID NO:1-4 include both PR and NPB sequences (PR+NPB). Immunogenic PspA polypeptides of SEQ ID NOS:5 and 6 include only a PR sequence (PR only) and lack the NPB.

Another appropriate art-recognized class of S. pneumoniae antigen are the pneumolysoids. Pneumolysoids have homology to the S. pneumoniae protein pneumolysin (PLY), but have reduced toxicity compared to pneumolysin. Pneumolysin is a key component in the pathogenesis of streptococcal pneumonia. The use of pneumolysin (or its homologues) as a part of a vaccine for S. pneumoniae lung infections and otitis media is becoming increasingly important due to the described drawbacks of typical CP-based strategies. A pneumolysin mutant (referred to as “Pd-B”) contains a single mutation at position 433 (wherein the native tryptophan residue has been changed to a phenylalanine). This mutation in pneumolysin is in the conserved undecapeptide of Domain 4, the structure within the cholesterol-dependent cytolysins (CDCs), which has long been thought to mediate binding to mammalian membranes. Other mutants of pneumolysin are described in U.S. Pat. No. 6,716,432, for example. Mutations are sought typically to provide an antigen with lower toxicity than native pneumolysin, but that still have a relative effective antigenicity so as to stimulate effective antibody response. See, e.g., U.S. Pat. No. 8,128,939.

Pneumolysoids encompassed by the present disclosure, thus, can be naturally occurring or engineered derivatives of pneumolysin. In some embodiments, a pneumolysoid has at least 70%, 80%, 85%, 90%, 95%, 97%, 98%, or 99% identity to the amino acid sequence of native pneumolysin, as set forth herein as SEQ ID NO:9, or a fragment thereof. In some embodiments, the pneumolysoid demonstrates less than ½, ⅕, 1/10, 1/20, 1/50, 1/100, 1/200, 1/500, or 1/1000 the toxicity of pneumolysin in an assay for one or both of hemolytic activity towards erythrocytes and inhibition of polymorphonuclear leukocytes. Both assays are described in Saunders et al., 1989, Infect. Immun. 57:2547-2552. Exemplary pneumolysoids include PdT (a triple mutant further described in Berry et al., 1995, Infect. Immun. 63:1969-1974); Pd-A and Pd-B (Paton et al., 1991, Infect. Immun. 59:2297-2304); rPd2 and rPd3 (Ferreira et al., 2006, FEMS Immunol. Med. Microbiol. 46:291-297); Ply8, δPLY, L460D (see, e.g., U.S. Pub. No. 2009/0285846 and L. Mitchell, Protective Immune Responses to Streptococcus pneumoniae Pneumolysoids, ASM2011 conference abstract, 2011), or a variant thereof. In some embodiments, the pneumolysin has a mutation in the catalytic domain, such as at amino acid 428 or 433 or the vicinity. In some embodiments, pneumolysin mutants can have substitutions at position 460, 370 and/or 406 of pneumolysin, as well as substitutions in one or more of three residues that flank either side of positions 370, 406 or 460, including positions 367, 368, 369, 371, 372, 373, 403, 404, 405, 407, 408, 409, 457, 458, 459, 461, 462, and 463. For example, these residues may be substituted with a negatively-charged amino acid, glutamate, or aspartate (except in position 403, which already comprises aspartate), or a positively charged amino acid lysine, arginine, or histidine (except in positions 367 and 407, which already comprise histidine residues). Alternatively, these residues may be substituted with any other natural amino acid (including gly, ala, leu, ile, val, pro, trp, asn, gin, phe, tyr, met, cys, thr, or ser) which abrogates the binding activity, pore-forming, and hemolytic activity of the mutant. See U.S. Pat. No. 8,128,939, incorporated herein by reference in its entirety.

Other appropriate S. pneumoniae antigens to serve as B-cell/antibody target antigens include PhtX, including PhtA, PhtB, PhtD, PhtE, Choline-binding proteins PcpA and CbpA and derivatives thereof (Ogunniyi et al., 2001. Infect. Immun. 69:5997-6003); caseinolytic protease; sortase A (SrtA); pilus 1 KrgA adhesin; PpmA; PrtA; PavA; LytA; Stk-PR; PcsB; and RrgB and derivatives thereof:

2. T_{H}17 Cell Antigens

As described above, the subset of T cells expressing IL-17A, i.e., T_{H}17 cells, have been identified as playing an important role in natural immunity to mucosal pathogens, such as S. pneumoniae. Accordingly, the vaccines and immunogenic compositions described herein include a polypeptide component directed to inducing a T_{H}17 response to S. pneumoniae. This component contributes the advantages of preventing or lowering mucosal colonization and carriage against a wide spectrum of S. pneumoniae serotypes.

Immunogenic polypeptide antigens that induce T_{H}17 cells can be identified according to known methods. Extensive genomic information for S. pneumoniae is known that can assist prediction of effective T cell antigens. For example, the S. pneumoniae ATCC 700669 complete genome sequence is available under GenBank accession number FM211187.1 (incorporated herein by reference) and polypeptide sequences are linked therein. Several known algorithms and computational tools can be used to predict immunogenicity of known or predicted polypeptide sequences, such as EpiMatrix (produced by EpiVax), PEPVAC (Promiscuous EPitope-based VACcine, hosted by Dana Farber Cancer Institute on the world wide web), MHCPred (which uses a partial least squares approach and is hosted by The Jenner Institute on the world wide web), and Immune Epitope Database algorithms available on the world wide web. Additionally, immunogenic portions can be identified by various methods, including protein microarrays, ELISPOT/ELISA techniques, and/or specific assays on different deletion mutants (e.g., fragments) of the polypeptide in question.

In one illustrative example, as reported in more detail in Moffitt, et al., 2011, Cell Host & Microbe, 9:158-165, incorporated herein by reference, a library containing 2207 of the predicted 2233 open reading frames predicted in the S. pneumoniae genome was cloned and expressed with an in-frame 1 cell epitope. The final validated library of expressed and MH-IC-presented peptides was estimated to reflect 95% of the total proteome sequence of S. pneumoniae. Macrophages presenting the S. pneumoniae peptides were screened against CD4⁺ T cells isolated from mice that had been immunized with a killed S. pneumoniae whole cell vaccine, and peptides inducing IL-17 expression (i.e., T_{H}17 induction) were identified. Bioinformatic analyses assisted the identification of top candidates by identifying amino acid sequences that had no homology with human proteins and low conservation with protein sequences of other bacteria. Additionally, to assess whether the identified antigens are well-presented during pneumococcal exposure, the IL-17A responses of experimentally colonized mice were evaluated in vitro by stimulating splenocytes isolated from mice previously intranasally inoculated with S. pneumoniae with purified antigens. Finally, the purified antigens were used to stimulate human PBMCs isolated from healthy adult donors to determine whether humans prime T₁₁17 cells specifically for the selected antigens during the course of natural exposure to S. pneumoniae.

Thus, polypeptide antigens indicated as effective antigens for stimulating T_{H}17 cells using approaches such as that described in Moffitt, et al., 2011, are useful in this aspect of the disclosure. Exemplary T_{H}17 polypeptide antigens are listed below in Table 2. These and additional T_{H}17 polypeptide antigens are listed in Tables 1 and 2 of U.S. Pub. No. US20120189649, incorporated herein by reference in its entirety.

TABLE 2
Exemplary immunogenic polypeptides useful for stimulating T_{H}17 cells.

| | Protein | |
|---|---|---|
| Locus tag name | SEQ ID | |
| and description | NO: | DNA GenGank Accession No. |
| SP0024 | 13 | NC_003028.3\|:27381-27878 |
| SP0882 | 14 | NC_003028.3\|:83 1804-832628 |
| SP0882N | 15 | |
| SP0882 with exogenous | 16 | |
| signal sequence | | |
| SP0882N with exogenous | 17 | |
| signal sequence | | |
| SP0148 lacking signal | 18 | |
| sequence | | |
| SP0148 including signal | 19 | NC_003028.3\|:145,513-146,343* |
| sequence | | |
| SP1072 | 20 | NC_003028.3\|:1008420-1010180 |
| SP2108 including signal | 21 | NC_003028.3\|:2020750-2022021 |
| sequence | | |
| SP2108 lacking signal | 22 | |
| sequence | | |
| SP0641M | 23 | |
| SP0641 | 24 | NC_003028.3\|:2020750-2022021 |
| SP0641N | 25 | |
| SP0882 consensus | 26 | |
| SP0882N consensus | 27 | |
| SP0882 consensus with | 28 | |
| exogenous leader | | |
| SP0882N consensus with | 29 | |
| exogenous leader | | |
| SP0148 consensus lacking | 30 | |
| signal sequence | | |
| SP0148 consensus including | 31 | |
| signal sequence | | |
| SP2108 consensus lacking | 32 | |
| signal sequence | | |
| SP2108 consensus including | 33 | |
| signal sequence | | |
| SP1634 | 34 | NC_003028.3\|:1534348-1535421 |
| SP0314 | 35 | NC_003028.3\|:287483-290683 |
| SP1912 | 36 | NC_003028.3\|:824672-1824971 |
| SP1912L | 37 | |
| SP0641.1 | 38 | |
| SP1912 consensus | 39 | |
| SP0641N consensus | 40 | |
| SP0641M consensus | 41 | |

*The database sequence incorrectly lists TTG (encoding Leu) at nucleotide positions 541-543. The correct sequence is TTC at that codon and encodes Phe.

3. Fusion Proteins

In some embodiments, one or more, e.g., two, three, four, or more polypeptides from Table 1 and/or Table 2 or immunogenic fragments or variants thereof are provided in a mixture. In some embodiments, the mixture contains both full-length polypeptides and/or fragments resulting from processing, or partial processing, of signal sequences by an expression host, e.g., E. coli, an insect cell line (e.g., the baculovirus expression system), or a mammalian (e.g., human or Chinese Hamster Ovary) cell line. In some embodiments, rather than being in a simple physical mixture, two, three, four, or more polypeptides from Table 1 and/or Table 2, or immunogenic fragments or variants thereof are covalently bound to each other, e.g., as a fusion protein.

Thus, in some embodiments, two or more of the antigens are fused or linked. For example, in some embodiments, the vaccine or immunogenic composition comprises fusion proteins. An exemplary fusion protein includes a first portion that primarily elicits a T-cell response and a second portion that primarily elicits a B-cell (e.g., antibody) response, or vice versa. In certain embodiments, the fusion proteins include one, two or more of the polypeptides (or genes) described herein as a B-cell antigen and a T-cell antigen listed in Table 1 and/or Table 2. In certain embodiments, the fusion protein includes a polypeptide or gene listed in Table 1 fused to a polypeptide or gene listed in Table 2.

In some embodiments, the fusion protein comprises an N-terminal peptide and a C-terminal peptide. In some embodiments, the N-terminal peptide comprises an immunogenic polypeptide that induces a T_{H}17 response, for example, any polypeptide described herein for this purpose. Some examples include the polypeptides having an amino acid sequence comprising SEQ ID NOS:13-41, or immunogenic fragments or variants thereof. In these embodiments, the C-terminal peptide can comprise an S. pneumoniae antigen that is predominantly an antibody target as described herein or immunogenic fragments or variants thereof. In other embodiments, the N-terminal peptide comprises an S. pneumoniae antigen that is predominantly an antibody target, as described herein, and the C-terminal peptide can comprise an immunogenic polypeptide that induces a T_{H}17 response, for example, the polypeptide having an amino acid sequence comprising SEQ ID NOS: 13-41, or immunogenic fragments or variants thereof.

In some embodiments, the antigenic peptides at the N-terminal and the C-terminal are directly bound to each other. In other embodiments, the antigenic peptides at the N-terminal and the C-terminal are linked via a linker peptide. The length of and/or amino acids that comprise a linker, when present, can be adjusted to obtain a more flexible or rigid linker. Exemplary peptide linkers are shown as SEQ ID NOS:42-44. A linker can generally be from 1-40, such as 10-30 and specifically 10, 11, 12, 13, 14, 15, 16, 17, 18, 19 or 20 amino acids in length.

An illustrative, nonlimiting list of potential fusion proteins is set forth in Table 3.

TABLE 3
Immunogenic fusion proteins for S. pneumoniae vaccine formulations.

| | DNA SEQ | Protein SEQ |
|---|---|---|
| Locus tag name | ID NO: | ID NO: |
| SP2108/SP0148 | 45 | 46 |
| SP0148/SP2108 | 47 | 48 |
| SP2108/SP1912 | 49 | 50 |
| SP0148/SP1912 | 51 | 52 |
| SP2108/SP1912/SP0148 | 53 | 54 |
| SP0148/SP1912/SP2108 | 55 | 56 |
| SP2108/SP0148/SP1912 | 57 | 58 |
| SP0148/SP2108/SP1912 | 59 | 60 |
| SP0148/CD2 | 61 | 62 |
| SP0148/H70 | 63 | 64 |
| SP2108/CD2 | 65 | 66 |
| SP2108/H70 | 67 | 68 |
| SP0148/LC/CD2 | 69 | 70 |
| SP0148/ LC/H70 | 71 | 72 |
| SP2108/LC/CD2 | 73 | 74 |
| SP2108/LC/H70 | 75 | 76 |
| SP0148/LR/CD2 | 77 | 78 |
| SP0148/LR/H70 | 79 | 80 |
| SP2108/LR/CD2 | 81 | 82 |
| SP2108/LR/H70 | 83 | 84 |

4. General Characteristics of Protein Antigens

As described, the vaccine and immunogenic compositions of the present disclosure incorporate a protein component with one or more polypeptides that serve as effective antigens for T cells and for B cells. Thus, the polypeptides of the present disclosure, and fragments and variants thereof, are immunogenic. This includes instances where the polypeptides are fused, mixed, or coordinately administered with other polypeptide or polysaccharide antigens, adjuvants, or carriers. These polypeptides can be immunogenic in mammals, for example mice, guinea pigs, or humans. An immunogenic polypeptide is typically one capable of raising a significant immune response in an assay or in a subject. The immune response can be innate, humoral, cell-mediated, and/or mucosal (combining elements of innate, humoral and cell-mediated immunity). For instance, an immunogenic polypeptide can induce the production of IL-17 produced by antigen-specific T cells. Alternatively or additionally, an immunogenic polypeptide can (i) induce production of antibodies, e.g., neutralizing antibodies, that bind to the polypeptide and/or the whole bacteria, (ii) induce T_{H}17 immunity, (iii) activate the CD4⁺ T cell response, for example by stimulating antigen-specific CD4⁺ T cells and/or increasing localization of CD4⁺ T cells to the site of infection or reinfection, (iv) activate the CD8⁺ T cell response, for example by increasing CD8⁺ T cells and/or increasing localization of CD8⁺ T cells to the site of infection or reinfection, (v) induce THil immunity, and/or (vi) activate innate immunity. In some embodiments, an immunogenic polypeptide causes the production of a detectable amount of antibody specific to that antigen.

In certain embodiments, polypeptides have less than 20%, 30%, 40%, 50%, 60% or 70% identity to human autoantigens and/or gut commensal bacteria (e.g., certain Bacteroides, Clostridium, Fusobacterium, Eubacterium, Ruminococcus, Peptococcus, Peptostreptococcus, Bifidobacterium, Escherichia and Lactobacillus species). Examples of human autoantigens include insulin, proliferating cell nuclear antigen, cytochrome P450, and myelin basic protein.

A polypeptide can comprise one or more immunogenic portions and one or more nonimmunogenic portions. The immunogenic portions can be identified by various methods, including protein microarrays, ELISPOT/ELISA techniques, T cell cloning and/or specific assays on different deletion mutants (e.g., fragments) of the polypeptide in question. Immunogenic portions can also be identified by computer algorithms. Some such algorithms, like EpiMatrix (produced by EpiVax), use a computational matrix approach. Other computational tools for identifying antigenic epitopes include PEPVAC (Promiscuous EPitope-based VACcine, hosted by Dana Farber Cancer Institute on the world wide web), MI ICPred (which uses a partial least squares approach and is hosted by The Jenner Institute on the world wide web), and Immune Epitope Database algorithms which is also available on the world wide web. An immunogenic fragment of a polypeptide described herein comprises at least one immunogenic portion, as measured experimentally or identified by algorithm.

Thus, in some aspects, this application provides an immunogenic fragment of an antigen described herein. The fragments, in some instances, are close in size to the full-length polypeptide or equivalent to the polypeptides listed in Table 1 or Table 2. For example, they can lack at most one, two, three, four, five, ten, twenty, or thirty amino acids from one or both termini. In certain embodiments, the polypeptide is 100-500 amino acids in length, or 150-450, or 200-400, or 250-250 amino acids in length. In some embodiments, the polypeptide is 100-200, 150-250, 200-300, 250-350, 300-400, 350-450, or 400-500 amino acids in length. In certain embodiments, the fragments result from processing, or partial processing, of signal sequences by an expression host, e.g., E. coli, an insect cell line (e.g., the baculovirus expression system), or a mammalian (e.g., human or Chinese Hamster Ovary) cell line. The fragments described above or sub-fragments thereof (e.g., fragments of 8-50, 8-30, or 8-20 amino acid residues) preferably have one of the biological activities described herein, such as inducing the production of IL-17. For example, the fragments can induce increased IL-17 production by at least 1.5 fold or 2 fold, or more (e.g., either as an absolute measure or relative to an immunologically inactive protein). A fragment can be used as the polypeptide in the vaccines described herein and can be fused to another protein, protein fragment or other antigen.

Individual strains of S. pneumoniae contain numerous mutations relative to each other, and some of these result in different protein sequences between the different strains. One of skill in the art can readily substitute an amino acid sequence, or a portion thereof, with the homologous amino acid sequence from a different S. pneumoniae strain for any amino acid antigen described herein. In certain aspects, this application encompasses immunogenic polypeptides with at least 90%, 95%, 97%, 98%, 99%, or 99.5% identity to the polypeptides of Table 1, 2, or 3, any other polypeptide antigen sequence described herein, or an immunogenic fragment thereof. Serotypic variation can be used to design such variants of the polypeptides of Tables 1, 2, and/or 3. In some cases, the polypeptide antigen is at least 70%, 80%, 85%, 90%, 95%, 97%, 98%, or 99% identical to the corresponding wild-type S. pneumoniae protein. Sequences of the polypeptides described herein, and nucleic acids that encode them, are known; see, for example, the S. pneumoniae ATCC 700669 complete genome sequence under GenBank accession number FM211187.1 and linked polypeptide sequences therein.

An immunogenic composition can also comprise portions of the Streptococcus polypeptides, including fusion proteins, for example deletion mutants, truncation mutants, oligonucleotides, and peptide fragments. In some embodiments, the portions of said polypeptides are immunogenic. The immunogenicity of a portion of a protein is readily determined using the same assays that are used to determine the immunogenicity of the full-length protein. In some embodiments, the portion of the polypeptide has substantially the same immunogenicity as the full-length proteins. In some embodiments, the immunogenicity is no more than 10%, 20%, 30%, 40%, or 50% less than that of the full-length protein (e.g., polypeptides of Table 1, 2, or 3, or otherwise described herein). The peptide fragments can be, for example, linear, circular, or branched.

In some embodiments, the fragment is a truncated fragment of any of SEQ ID NOS:1-41, 46, 48, 50, 52, 54, 56, 58, 60, 62, 64, 66, 68, 70, 72, 74, 76, 78, 80, 82, and 84, having from 1-5, 1-10, or 1-20 amino acid residues removed from the N-terminus, C-terminus, or both. In some such embodiments, the same number of residues is removed from the N-terminus and the C-terminus, while in other embodiments, a different number of residues is removed from the N-terminus compared to the C-terminus.

Some embodiments of the vaccine formulations and immunogenic compositions described herein include an immunogenic polypeptide, including fusion proteins, (e.g., a polypeptide of Table 1, 2, or 3, or otherwise described herein) that contains a membrane translocating sequence (MTS), to facilitate introduction of the polypeptide into the mammalian cell and subsequent stimulation of the cell-mediated immune response. Exemplary membrane translocating sequences include a hydrophobic region in the signal sequence of Kaposi fibroblast growth factor, the MTS of α-synuclein, β-synuclein, or γ-synuclein, the third helix of the Antennapedia homeodomain, SN50, integrin β3 h-region, HIV Tat, pAntp, PR-39, abaecin, apidaecin, Bac5, Bac7, P. berghei CS protein, and those MTSs described, for example, in U.S. Pat. Nos. 6,248,558, 6,432,680, and 6,248,558.

In certain embodiments, an antigen (e.g., a polypeptide of Tables 1, 2, or 3, or a fragment thereof) is covalently bound to another molecule. This can, for example, increase the half-life, solubility, bioavailability, or immunogenicity of the antigen. Molecules that can be covalently bound to the antigen include a carbohydrate, biotin, poly(ethylene glycol) (PEG), polysialic acid, N-propionylated polysialic acid, polysaccharides, and PLGA. There are many different types of PEG, ranging from molecular weights of below 300 g/mol to over 10,000,000 g/mol. PEG chains can be linear, branched, or with comb or star geometries. In some embodiments, the naturally produced form of a protein is covalently bound to a moiety that stimulates the immune system. An example of such a moiety is a lipid moiety. In some instances, lipid moieties are recognized by a Toll-like receptor (TLR) such as TLR-2 or TLR-4, and activate the innate immune system.

In certain embodiments, vaccines or pharmaceutical compositions comprising an S. pneumoniae polypeptide, including a fusion protein comprising an S. pneumoniae polypeptide, contain at least one lipidated polypeptide. In some embodiments, the protein or fusion protein is lipidated. In certain embodiments, the protein or fusion protein is lipidated on the N-terminal peptide. Conjugation to the lipid moiety can be direct or indirect (e.g., via a linker). The lipid moiety can be synthetic or naturally produced. In certain embodiments, a polypeptide from Table 1, 2, or 3 can be chemically conjugated to a lipid moiety. In certain embodiments, a construct can comprise a gene or polypeptide from Table 1, 2, or 3, or an immunogenic fragment or variant thereof, and a lipidation sequence including a lipobox motif. A canonical lipobox motif is shown as SEQ ID NO:85. A lipidation sequence can be N-terminal or C-terminal to the protein, and can be embedded in a signal or other sequence, or in a fusion protein. Exemplary lipidation sequences include the signal sequence of SP2108 (SEQ ID NO:86) and the signal sequence of the E. coli gene RlpB (SEQ ID NO:87). A signal sequence can be, for example, an E. coli or S. pneumoniae signal sequence. Exemplary E. coli signal sequences include the mlpA signal sequence (Lin et al., 1978, Proc. Natl. Acad. Sci. U.S.A. 75:4891-4895), the lamB signal sequence (Emr et al., 1978, Proc. Natl. Acad. Sci. U.S.A. 75:5802-5806), the MBP signal sequence (Bassford, 1979, J. Bacteriol. 139:19-31). Lpp is an exemplary E. coli signal sequence that directs lipidation (Cullen et al., 2003, Plasmid. 49:18-29.) E. coli signal sequences that direct lipidation are also described in Legrain et al., 1995, Protein Expr. Purif 6:570-578), e.g., the signal sequence of the gene RlpB (SEQ ID NO:87). Numerous S. pneumoniae signal sequences are known in the art. One such signal sequence is SEQ ID NO:86.

In certain embodiments, vaccines or pharmaceutical compositions comprising an S. pneumoniae polypeptide including a fusion protein can comprise a polypeptide from Table 1 and/or 2, or an immunogenic fragment or variant thereof, and a tag. A tag can be N-terminal or C-terminal. For instance, tags can be added to the polypeptide to facilitate purification, detection, solubility, or confer other desirable characteristics on the protein. For instance, a purification tag can be a peptide, oligopeptide, or polypeptide that can be used in affinity purification. Examples include His, GST, TAP, FLAG, myc, HA, MBP, VSV-G, thioredoxin, V5, avidin, streptavidin, BCCP, Calmodulin, Nus, S tags, lipoprotein D, and β galactosidase. Particular exemplary His tags include HIHHHHH (SEQ ID NO:88) and MSYYHHHHHHH (SEQ ID NO:89). In other embodiments, the polypeptide is free of tags such as protein purification tags, and is purified by a method not relying on affinity for a purification tag. In some embodiments, the fused portion is short. This, in some instances, the fusion protein comprises no more than 1, 2, 3, 4, 5, 10, or 20 additional amino acids on one or both termini of polypeptide from Table 1 and/or 2.

### C. Additional Components of Vaccine and Immunogenic Compositions

In certain embodiments, the vaccine or immunogenic composition comprises a plurality of antigens described herein and one or more of the following: an adjuvant (e.g., a vaccine delivery system and/or immunostimulatory compound), stabilizer, buffer, surfactant, controlled release component, salt, and/or a preservative.

1. Carriers/Adjuvants

The vaccine formulations and immunogenic compositions described herein can include an adjuvant. Adjuvants can be broadly separated into two classes, based on their principal mechanisms of action: vaccine delivery systems and immunostimulatory compounds (see, e.g., Singh et al., Curr. HIV Res. 1:309-320, 2003).

Vaccine delivery systems are often advantageously particle formulations. Examples of particle formulations include emulsions, microparticles, immune-stimulating complexes (ISCOMs), nanoparticles, which can be, for example, particles and/or matrices, and liposomes, and the like. Such formulations are often effective for delivery of intact antigens, or collections of different antigenic vaccine components that work in concert, to promote robust immune responses.

Oil emulsion compositions suitable fbor use as adjuvants/carriers in the present disclosure include squalene-water emulsions, such as MF59 (5% Squalene, 0.5% Tween 80, and 0.5% Span 85, formulated into submicron particles using a micro fluidizer). See, e.g., WO2009016515.

Microparticles (i.e., a particle of ˜100 nm to ˜150 μm in diameter, more preferably ˜200 nm to ˜30 μm in diameter, and most preferably ˜500 nm to ˜10 μm in diameter) can be formed from materials that are biodegradable and non-toxic (e.g., a poly(α-hydroxy acid), a polyhydroxybutyric acid, a polyorthoester, a polyanhydride, a polycaprolactone, etc.). Poly(lactide-co-glycolide)-based microparticles are known as effective carriers, which are optionally treated to have a negatively-charged surface (e.g., with SDS) or a positively-charged surface (e.g., with a cationic detergent, such as CTAB).

Nanoparticles have been shown to be effective delivery vehicles for vaccine compositions and can enhance the immune response. Individual nanoparticles are typically between 1 and 100 nanometers. In some embodiments, the nanoparticles are solid and can have the vaccine components conjugated to the surface. In other embodiments, the nanoparticles, such as liposomes, can be formed around and thus encapsulating mixtures of vaccine components. Liposome vesicles, for example, can be formed around the vaccine components using pure phospholipid or a mixture of phospholipids and phosphoglycerides according to known methods. See, e.g., U.S. Pub. No. 2006/0189554. In some embodiments, protein antigens, such as T_{H}17 cell protein antigen or B cell protein antigen, can be lapidated to facilitate their incorporation into the nanoparticle. Moreover CP antigens can be conjugated to the lipid nanoparticle coats according to known methods.

In addition, or alternatively, an adjuvant is provided to generate a signal to the immune system so that it generates a response to the antigen, wherein the antigen drives the specificity of the response to the pathogen. Such “immunostimulatory” compound adjuvants are sometimes derived from pathogens and can represent pathogen associated molecular patterns (PAMP), e.g., lipopolysaccharides (LPS), monophosphoryl lipid (MPL), or CpG-containing DNA, which activate cells of the innate immune system.

Such immunostimulatory compound adjuvants can be classified as organic and inorganic. Preferred inorganic adjuvants include aluminum salts (alum) such as aluminum phosphate, amorphous aluminum hydroxyphosphate sulfate, and aluminum hydroxide, which are commonly used in human vaccines and are easily adapted to new vaccine technologies.

Organic adjuvants comprise organic molecules including macromolecules. An example of an organic adjuvant is cholera toxin.

Known adjuvants can also be selected on the basis of the response they induce. In some embodiments, the adjuvant induces the activation of T_{H}1 cells or T_{H}2 cells. In other embodiments, the adjuvant induces the activation of B cells. In yet other embodiments, the adjuvant induces the activation of antigen-presenting cells. These categories are not mutually exclusive; in some cases, an adjuvant activates more than one type of cell.

In certain embodiments, the adjuvant can induce the activation of T_{H}17 cells. It can promote the CD4⁺ or CD8⁺ T cells to secrete IL-17. In some embodiments, an adjuvant that induces the activation of T_{H}17 cells is one that produces at least a 2-fold, and in some cases a 10-fold, experimental sample-to-control ratio in the following assay. In the assay, an experimenter compares the IL-17 levels secreted by two populations of cells: (1) cells from animals immunized with the adjuvant and a polypeptide known to induce T_{H}17 activation, and (2) cells from animals treated with the adjuvant and an irrelevant (control) polypeptide. An adjuvant that induces the activation of T_{H}17 cells can cause the cells of population (1) to produce more than 2-fold, or more than 10-fold more IL-17 than the cells of population (2). IL-17 can be measured, for example, by ELISA or ELISPOT. Certain toxins, such as cholera toxin and labile toxin (produced by enterotoxigenic E. coli, or ETEC), activate a T_{H}17 response. Thus, in some embodiments, the adjuvant is a toxin. One form of labile toxin is produced by Intercell. Mutant derivates of labile toxin that are active as adjuvants but significantly less toxic can be used as well. Exemplary detoxified mutant derivatives of labile toxin include mutants lacking ADP-ribosyltransferase activity. Particular detoxified mutant derivatives of labile toxin include LTK7 (Douce et al, 1995, Proc. Natl. Acad. Sci. USA 92:1644-1648) and LTK63 (Williams et al., 2004, J. Immunol. 173:7435-7443), LT-G192 (Douce et al., 1999, Infect. Immun. 67:4400-4406), and LTR72 (Giuliani et al., 1998, J. Exp. Med. 187:1123-1132).

In some embodiments, the adjuvant comprises a VLP (virus-like particle). One such adjuvant platform, Alphavirus replicons, induces the activation of T_{H}17 cells using alphavirus and is produced by Alphavax. In certain embodiments of the Alphavirus replicon system, alphavirus can be engineered to express an antigen of interest, a cytokine of interest (for example, IL-17 or a cytokine that stimulates IL-17 production), or both, and can be produced in a helper cell line. More detailed information can be found in U.S. Pat. No. 5,643,576 and U.S. Pat. No. 6,783,939.

In some embodiments, a vaccine formulation is administered to a patient in combination with a nucleic acid encoding a cytokine. Certain classes of adjuvants activate toll-like receptors (TLRs) in order to activate a T_{H}17 response. TLRs are well known proteins that can be found on leukocyte membranes, and recognize foreign antigens (including microbial antigens). Administering a known TLR ligand together with an antigen of interest (for instance, as a fusion protein) can promote the development of an immune response specific to the antigen of interest. One exemplary adjuvant that activates TLRs comprises Monophosphoryl Lipid A (MPL). Traditionally, MPL has been produced as a detoxified lipopolysaccharide (LPS) endotoxin obtained from gram negative bacteria, such as S. minnesota. In particular, sequential acid and base hydrolysis of LPS produces an immunoactive lipid A fraction (which is MPL), and lacks the saccharide groups and all but one of the phosphates present in LPS. A number of synthetic TLR agonists (in particular, TLR-4 agonists) are disclosed in Evans et al., 2003, Expert Rev. Vaccines 2:219-229. Like MPL adjuvants, these synthetic compounds activate the innate immune system via TLR-4. Another type of TLR agonist is a synthetic phospholipid dimer, for example E6020 (Ishizaka et al., 2007, Expert Rev. Vaccines 6:773-784). Various TLR agonists (including TLR-4 agonists) have been produced and/or sold by, for example, the Infectious Disease Research Institute (IRDI), Corixa, Esai, Avanti Polar Lipids, Inc., and Sigma Aldrich. Another exemplary adjuvant that activates TLRs comprises a mixture of MPL, Trehalose Dicoynomycolate (TDM), and dioctadecyldimethylammonium bromide (DDA). Another TLR-activating adjuvant is R848 (resiquimod).

In some embodiments, the adjuvant is or comprises a saponin. Typically, the saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C.

A particular form of saponins that can be used in vaccine formulations described herein is immunostimulating complexes (ISCOMs). ISCOMs are an art-recognized class of adjuvants, that generally comprise Quillaja saponin fractions and lipids (e.g., cholesterol and phospholipids such as phosphatidyl choline). In certain embodiments, an ISCOM is assembled together with a polypeptide of interest. However, different saponin fractions can be used in different ratios. In addition, the different saponin fractions can either exist together in the same particles or have substantially only one fraction per particle (such that the indicated ratio of fractions A and C are generated by mixing together particles with the different fractions). In this context, “substantially” refers to less than 20%, 15%, 10%, 5%, 4%, 3%, 2% or even 1%. Such adjuvants can comprise fraction A and fraction C mixed into a ratio of 70-95 A:30-5 C, such as 70 A:30 C to 75 A:5 C, 75 A:5 C to 80 A:20 C, 80 A:20 C to 85 A:15C, 85 A:15 C to 90 A:10 C, 90 A:10 C to 95 A:5 C, or 95 A:5 C to 99 A:1 C.

In certain embodiments, combinations of adjuvants are used. For example, in some embodiments an adjuvant that promotes a B cell response to the protein and/or CP antigens, such as aluminum, can be combined with an adjuvant that promotes a T cell response, such as TLR agonists. Three exemplary combinations of adjuvants are MPL and alum, E6020 and alum, and MPL and an ISCOM.

An adjuvant can be covalently bound to an antigen. In some embodiments, the adjuvant can comprise a protein which induces inflammatory responses through activation of antigen presenting cells (APCs). In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to naïve T cells and initiation of T cell responses (see for example, Wu et al., 2005, Cancer Res. 65:4947-4954). In certain embodiments, a polypeptide, including a fusion protein, described herein is presented in the context of the trivalent conjugate system, comprising a fusion protein of S. pneumoniae Pneumococcal surface adhesin A (PsaA) with the pneumolysoid PdT and a cell wall polysaccharide (PsaA:PdT-CPs), described in Lu et al., 2009, Infect. Immun. 77:2076-2083. The pneumolysin derivative PdT carries three amino acid substitutions (W433F, D385N, and C428G) which render the molecule nontoxic but do not interfere with its TLR-4-mediated inflammatory properties. Conjugation of a polysaccharide to the fusion of a polypeptide to the TLR-4-agonist PdT enhances immunological response to the polypeptide. In some embodiments, one or more polypeptides described herein are used in place of PsaA in the trivalent conjugate. The trivalent conjugate system typically includes alum and is usually administered parenterally. Other exemplary adjuvants that can be covalently bound to antigens comprise polysaccharides, pneumolysin, synthetic peptides, lipopeptides, and nucleic acids.

Typically, the same adjuvant or mixture of adjuvants is present in each dose of a vaccine. Optionally, however, an adjuvant can be administered with the first dose of vaccine and not with subsequent doses (i.e., booster shots). Alternatively, a strong adjuvant can be administered with the first dose of vaccine and a weaker adjuvant or lower dose of the strong adjuvant can be administered with subsequent doses. The adjuvant can be administered before the administration of the antigen, concurrent with the administration of the antigen or after the administration of the antigen to a subject (sometimes within 1, 2, 6, or 12 hours, and sometimes within 1, 2, or 5 days). Certain adjuvants are appropriate for human patients, non-human animals, or both.

2. Additional Components

In addition to the antigens and the adjuvants described above, a vaccine formulation or immunogenic composition can include one or more additional components, such as a stabilizer, buffer, surfactant, controlled release component, salt, and/or preservative.

In certain embodiments, the vaccine formulation or immunogenic composition can include one or more stabilizers such as sugars (such as sucrose, trehalose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In certain embodiments, the vaccine formulation or immunogenic composition includes one or more buffers such as a mixture of sodium bicarbonate and ascorbic acid. In some embodiments, the vaccine formulation can be administered in saline, such as phosphate buffered saline (PBS), or distilled water.

In certain embodiments, the vaccine formulation or immunogenic composition includes one or more surfactants such as, for example, polysorbate 80 (Tween 80), Triton X-100, Polyethylene glycol tert-octylphenyl ether t-Octylphenoxypolyethoxyethanol 4-(1,1,3,3-Tetramethylbutyl)phenylpolyethylene glycol (TRITON X-100); Polyoxyethylenesorbitan monolaurate Polyethylene glycol sorbitan monolaurate (TWEEN 20); 4-(1,1,3,3-Tetramethylbutyl)phenol polymer with formaldehyde and oxirane (TYLOXAPOL); and the like. A surfactant can be ionic or nonionic.

In certain embodiments, the vaccine formulation or immunogenic composition includes one or more salts such, for example, as sodium chloride, ammonium chloride, calcium chloride, or potassium chloride.

In certain embodiments, a preservative is included in the vaccine or immunogenic composition. In other embodiments, no preservative is used. A preservative is most often used in multi-dose vaccine vials, and is less often needed in single-dose vaccine vials. In certain embodiments, the preservative is, for example, 2-phenoxyethanol, methyl and propyl parabens, benzyl alcohol, thiomersal, and/or sorbic acid.

In certain embodiments, the vaccine formulation or immunogenic composition is a controlled release formulation.

### D. Use of Vaccines and Immunogenic Compositions

The S. pneumoniae vaccines described herein can be used for prophylactic and/or therapeutic treatment of S. pneumoniae. Accordingly, this application provides a method for treating a subject suffering from or susceptible to S. pneumoniae infection, comprising administering an effective amount of any of the vaccine formulations described herein. In some aspects, the method inhibits S. pneumoniae colonization in an individual. In some aspects, the method reduces or prevents nasopharyngeal carriage in an individual. In some aspects, the method inhibits S. pneumoniae symptoms, invasive disease or sequelae, such as sepsis, pneumonia, meningitis, otitis media, sinusitis or infection of other sites or organs with S. pneumoniae. The subject receiving the vaccination can be a male or a female, and can be a child or adult. In some embodiments, the subject being treated is a human. In other embodiments, the subject is a non-human animal.

1. Prophylactic Use

In prophylactic embodiments, the vaccine is administered to a subject to induce an immune response that can help protect against the establishment of S. pneumoniae, for example by protecting against colonization, the first and necessary step in disease progression. Thus, in some aspects, the method inhibits infection by S. pneumoniae in a non-colonized or uninfected subject. In another aspect, the method can reduce or eliminate the nasopharyngeal carriage by an individual. In another aspect, the method can reduce the duration of colonization in an individual who is already colonized.

In some embodiments, the vaccine compositions of the present disclosure confer protective immunity, allowing a vaccinated individual to exhibit delayed onset of symptoms or sequelae, or reduced severity of symptoms or sequelae, as the result of his or her exposure to the vaccine. In certain embodiments, the reduction in severity of symptoms or sequelae is at least 25%, 40%, 50%, 60%, 70%, 80%, or even 90%. In particular embodiments, vaccinated individuals can display no symptoms or sequelae upon contact with S. pneumoniae, do not become colonized by S. pneumoniae, or demonstrate reduced colonization/duration of colonization. Protective immunity is typically achieved by one or more of the following mechanisms: mucosal, humoral, and/or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes, that result in sIGA production by B lymphocytes on mucosa-lined tissues of the body. Humoral immunity is typically the result of IgG antibodies and IgM antibodies in serum. Cellular immunity can be achieved through cytotoxic T lymphocytes or through delayed-type hypersensitivity that involves macrophages and T lymphocytes, as well as other mechanisms involving T cells without a requirement for antibodies. In particular, cellular mucosal immunity can be mediated by T_{H}1 or T_{H}17 cells.

Essentially any individual has a certain risk of becoming infected with S. pneumoniae. However, certain sub-populations have an increased risk of infection. In some embodiments, a vaccine formulation as described herein (e.g., a composition comprising one or more polysaccharides antigens and one or more polypeptides capable of inducing a B cell and T cell response) is administered to patients that are immunocompromised.

An immunocompromising condition arising from a medical treatment is likely to expose the individual in question to a higher risk of infection with S. pneumoniae. It is possible to treat an infection prophylactically in an individual having the immunocompromised condition before or during treatments known to compromise immune function. By prophylactically treating with an antigenic composition (e.g., including one or more polypeptide antigens capable of inducing a B cell and/or T cell response, and one or more polysaccharide antigens), before or during a treatment known to compromise immune function, it is possible to prevent a subsequent S. pneumoniae infection or to reduce the risk of the individual contracting an infection due to the immunocompromised condition. Should the individual contract an S. pneumoniae infection e.g., following a treatment leading to an immunocompromised condition it is also possible to treat the infection by administering to the individual an antigen composition.

The following groups are at increased risk of pneumococcal disease or its complications, and therefore it is advantageous for subjects falling into one or more of these groups to receive a vaccine formulation described herein: children, especially those from 1 month to 5 years old or 2 months to 2 years old; children who are at least 2 years of age with asplenia, splenic dysfunction or sickle-cell disease; children who are at least 2 years of age with nephrotic syndrome, chronic cerebrospinal fluid leak, HIV infection or other conditions associated with immunosuppression.

In another embodiment, at least one dose of the pneumococcal combined antigen composition is given to adults in the following groups at increased risk of pneumococcal disease or its complications: all persons 65 years of age; adults with asplenia, splenic dysfunction or sickle-cell disease; adults with the following conditions: chronic cardiorespiratory disease, cirrhosis, alcoholism, chronic renal disease, nephrotic syndrome, diabetes mellitus, chronic cerebrospinal fluid leak, HIV infection, AIDS and other conditions associated with immunosuppression (e.g., Hodgkin's disease, lymphoma, multiple myeloma, immunosuppression for organ transplantation), individuals with cochlear implants; individuals with long-term health problems such as heart disease and lung disease, as well as individuals who are taking any drug or treatment that lowers the body's resistance to infection, such as long-term steroids, certain cancer drugs, radiation therapy; Alaskan natives and certain Native American populations.

2. Therapeutic Use

In therapeutic applications, the vaccine can be administered to a patient suffering from S. pneumoniae infection, in an amount sufficient to treat the patient. Treating the patient, in this case, refers to reducing S. pneumoniae symptoms and/or bacterial load and/or sequelae in an infected individual. Some individuals remain asymptomatic upon colonization but can carry a mucosal infection that can be transmitted to other individuals. Accordingly, in some embodiments, treatment refers to eliminating or reducing the mucosal bacterial load, or to reducing the duration of nasopharyngeal carriage. In some embodiments, treating the patient refers to reducing the infectivity of the patient to other individuals. In some embodiments, treating the patient refers to reducing the duration of symptoms or sequelae. In some embodiments, treating the patient refers to reducing the intensity of symptoms or sequelae. In some embodiments, the vaccine reduces transmissibility of S. pneumoniae from the vaccinated patient. In certain embodiments, the reductions described above are at least 25%, 30%, 40%, 50%, 60%, 70%, 80%, or even 90%.

In therapeutic embodiments, the vaccine is administered to an individual post-infection. The vaccine can be administered shortly after infection, e.g., before symptoms or sequelae manifest, or can be administered during or after manifestation of symptoms or sequelae.

A therapeutic S. pneumoniae vaccine can reduce the intensity and/or duration of the various symptoms or sequelae of S. pneumoniae infection. Symptoms or sequelae of S. pneumoniae infection can take many forms. In some cases, an infected patient develops pneumonia, acute sinusitis, otitis media (ear infection), meningitis, bacteremia, sepsis, osteomyelitis, septic arthritis, endocarditis, peritonitis, pericarditis, cellulitis, or brain abscess.

Sepsis is a rare but life-threatening complication of S. pneumoniae infection, where the bacterium invades the bloodstream and systemic inflammation results. Typically, fever is observed and white blood cell count increases. A further description of sepsis is found in Goldstein, B., et al., 2005, Pediatr. Crit. Care Med. 6:2-8.

3. Assaying Vaccination/Immunogenic Composition Efficacy

The efficacy of the vaccines and immunogenic compositions disclosed herein can be determined in a number of ways, in addition to the clinical outcomes described above. First, one can assay IL-17 levels (particularly IL-17A) by stimulating T cells derived from the subject after administration/vaccination. The IL-17 levels can be compared to IL-17 levels in the same subject before vaccination. Increased IL-17 (e.g., IL-17A) levels, such as a 1.5 fold, 2-fold, 5-fold, 10-fold, 20-fold, 50-fold 20 or 100-fold or more increase, would indicate an increased response to the vaccine. Alternatively (or in combination), one can assay neutrophils in the presence of T cells or antibodies from the patient for pneumococcal killing. Increased pneumococcal killing, such as a 1.5 fold, 2-fold, 5-fold, 10-fold, 20-fold, 50-fold, or 100-fold or more increase, would indicate an increased response to the vaccine. In addition, one can measure T_{H}17 cell activation, where increased T_{H}17 cell activation, such as a 1.5-fold, 2-fold, 5-fold, 10-fold, 20-fold, 50-fold or 100-fold or more increase, correlates with an increased response to the vaccine or immunogenic compositions. One can also measure levels of an antibody specific to the vaccine, where increased levels of the specific antibody, such as a 1.5-fold, 2-fold, 5-fold, 10-fold, 20-fold, 50-fold, or 100-fold or more increase, are correlated with increased vaccine or immunogenic composition efficacy. In certain embodiments, two or more of these assays are used. For example, one can measure IL-17 levels and the levels of vaccine-specific antibody. Alternatively, one can follow epidemiological markers such as incidence of, severity of, or duration of pneumococcal infection in vaccinated individuals compared to unvaccinated individuals.

The efficacy of the B-cell-directed antigen can be tested by measuring antibody titers against the B cell protein antigen, such as PspA or L460D pneumolysin or CP according to known assays. Some additional illustrative assays include testing for pneumolysin toxin neutralizing antibody responses, opsonophagocytic assay responses, and passive transfer of protection to mice against challenge with S. pneumoniae. These tests have the advantage establishing the quality of the antibody response, i.e., the efficacy of the antibodies produced in actually combating the S. pneumoniae, as opposed to merely establishing elevated levels of antibodies with unknown efficacy.

Vaccine efficacy can also be assayed in various model systems such as mouse models for pneumococcal carriage or disease. For instance, BALB/c or C57BL/6 strains of mice can be used. After administering the test vaccine to a subject (as a single dose or multiple doses), a challenge dose of S. pneumoniae is administered. In some cases, a challenge dose administered intranasally is sufficient to cause S. pneumoniae colonization (especially nasal colonization) in an unvaccinated animal, and in some cases a challenge dose administered via aspiration is sufficient to cause sepsis and a high rate of lethality in unvaccinated animals. One can then measure the reduction in colonization or the reduction in lethality in vaccinated animals.

Vaccine efficacy for specifically preventing or reducing nasopharyngeal carriage can be also assayed in available model systems as described above. In some embodiments, after administering the test vaccine to a model animal (in a single dose or multiple doses), at least one challenge dose of S. pneumoniae is administered intranasally, where the dose is sufficient to cause mucosal colonization in an unvaccinated animal. After sufficient time to allow establishment, the nasopharyngeal chamber is flushed and bacterial levels assayed from the obtained wash solution (e.g., CFU resulting per volume wash or using quantitative polymerase chain reaction (PCR) techniques directed to known S. pneumoniae genes). Furthermore, nasopharyngeal carriage can be monitored in humans receiving administrations of the described vaccine compositions. For example, carriage or carriage load can be assayed in individuals by obtaining mucosal samples using a deep nasopharyngeal swab technique. In one embodiment, a sterile swab with a flexible aluminum shaft and a dry calcium alginate tip is inserted into the nostril, and passed into the nasopharynx to a distance equal to that from the subject's nose to the tip of the ear. The sample can be stored in an appropriate medium, such as skim milk-tryptone-glucose-glycerol (STGG) medium, for quantification and/or identification assays. Nasopharyngeal carriage (i.e., presence of S. pneumoniae in the nasopharyngeal chamber) or carriage load can be assayed by known methods, including direct culturing techniques, detection of fluorescently labeled bacteria, quantitative PCR techniques directed to S. pneumnoniae genes, or commercially available kits such as BinaxNOW® (Alere, Waltham, Mass.).

4. Use of Immunogenic Compositions Against S. pneumoniae Infection

The immunogenic compositions of the present disclosure are designed to elicit an immune response against S. pneumoniae. Compositions described herein (e.g., ones comprising one or more polypeptides, including fusion proteins, and one or more polysaccharide antigens) can stimulate an antibody response or a cell-mediated immune response, or both, in the mammal to which it is administered. In some embodiments, the composition stimulates a T_{H}1-biased CD4⁺ T cell response, a T_{H}17-biased CD4⁺ T cell response and/or a CD8⁺ T cell response. In some embodiments, the composition stimulates an antibody response. In some embodiments, the composition stimulates a T_{H}1-biased CD4⁺ T cell response, T_{H}17-biased CD4⁺ T cell response and/or a CD8⁺ T cell response, and an antibody response.

In certain embodiments, the composition (e.g., one comprising one or more polypeptides, including fusion proteins, and one or more polysaccharide antigens) includes a cytokine such as IL-17, to provide additional stimulation to the immune system of the mammal.

While not wishing to be bound by theory, in some embodiments a T_{H}17 cell response is desirable in mounting an immune response to the compositions disclosed herein, e.g., ones comprising one or more polypeptides, including fusion proteins, and one or more polysaccharide antigens. In certain embodiments, an active T_{H}17 response is beneficial in clearing a pneumococcal infection. For instance, mice lacking the IL-17A receptor show decreased whole cell vaccine-based protection from a pneumococcal challenge (Lu et al., 2008, PLoS Pathog. 4.9:e1000159).

Thus, provided herein is a method of increasing IL-17 production by administering the compositions described herein (e.g., ones comprising one or more polypeptides described herein) to a subject. Furthermore, this application provides a method of activating T_{H}17 cells by administering said compositions to a subject. In certain embodiments, increased IL-17A levels contribute to increased pneumococcal killing by neutrophils or neutrophil-like cells, for instance by inducing recruitment and activation of neutrophils of neutrophil-like cells. In certain embodiments, this pneumococcal killing is independent of antibodies and complement. However, specific antibody production and complement activation can be useful additional mechanisms that contribute to clearing of a pneumococcal infection.

Immunogenic compositions containing immunogenic polypeptides and one or more lipid polysaccharides, together with a pharmaceutical carrier are also provided.

### E. Doses/Routes of Administration/Formulation

1. Dosage Forms, Amounts, and Timing

The amount of antigen in each vaccine or immunogenic composition dose is selected as an effective amount, which induces a prophylactic or therapeutic response against one, more or all of the antigens presented, as described above, in either a single dose or over multiple doses. Preferably, the dose is without significant adverse side effects in typical vaccines. Such amount will vary depending upon which specific antigens are employed. Generally, it is expected that a dose will comprise 1-100 μg of each protein antigen in the polypeptide component, for instance between 1-10 μg of each protein antigen. In some embodiments, the vaccine formulation comprises 1-250 μg, such as 1-100 μg, of the total polypeptide component; 1-250 μg, such as 1-100 μg, of the total CP antigen component; and 1-250 μg, such as 1-100 μg, of the adjuvant/carrier component. In some embodiments, the appropriate amount of protein and/or CP antigen component to be delivered will depend on the age, weight, and health (e.g., immunocompromised status) of a subject. When present, typically an adjuvant will be present in amounts from 1 μg-250 μg per dose, for example 50-150 μg, 75-125 μg or 100 μg.

In some embodiments, only one dose of the vaccine is administered to achieve the results described above. In other embodiments, following an initial vaccination, subjects receive one or more boost vaccinations, for a total of two, three, four or five vaccinations. Advantageously, the number is three or fewer. A boost vaccination can be administered, for example, about 1 month, 2 months, 4 months, 6 months, or 12 months after the initial vaccination, such that one vaccination regimen involves administration at 0, 0.5-2, and 4-8 months. It can be advantageous to administer split doses of vaccines which can be administered by the same or different routes. The vaccines and immunogenic compositions described herein can take on a variety of dosage forms. In certain embodiments, the composition is provided in solid or powdered (e.g., lyophilized) form; it also can be provided in solution form. In certain embodiments, a dosage form is provided as a dose of lyophilized composition and at least one separate sterile container of diluent or adjuvant.

In some embodiments, the composition, or discrete component thereof, (e.g., CP, B cell peptide antigen, and/or T cell peptide antigen) will be administered in a dose escalation manner in subsequence administrations, such that successive administrations of the composition contain a higher concentration of composition than previous administrations. In some embodiments, the composition will be administered in a manner such that successive administrations of the composition contain a lower concentration of composition than previous administrations.

In therapeutic applications, compositions are administered to a patient suffering from a disease in an amount sufficient to treat the patient. Therapeutic applications of a composition described herein include reducing transmissibility, slowing disease progression, reducing bacterial viability or replication, or inhibiting the expression of proteins required for toxicity, such as by 90%, 80%, 70%, 60%, 50%, 40%, 30%, 20%, or 10% of the levels at which they would occur in individuals who are not treated with the composition.

In prophylactic embodiments, compositions are administered to a human or other mammal to induce an immune response that can inhibit the establishment of an infectious disease or other condition. In some embodiments, a composition can partially block the bacterium from establishing an infection.

In some embodiments, the compositions are administered in combination with antibiotics. This co-administration is particularly appropriate when the pharmaceutical composition is administered to a patient who has recently been exposed (or is suspected of having been recently exposed) to S. pneumoniae. Many antibiotics are used to treat pneumococcal infections, including penicillin, amoxicillin, amoxicillin/clavulanate, cefuroxime, cefotaxime, ceftriaxone, and vancomycin. The appropriate antibiotic can be selected based on the type and severity of the infection, as well as any known antibiotic resistance of the infection (Jacobs, 1999, Am. J. Med. 106:19S-25S).

2. Routes of Administration

The vaccine formulations and pharmaceutical compositions herein can be delivered by administration to an individual, typically by systemic administration (e.g., intramuscular, intradermal, subcutaneous, subdermal, transdermal, intravenous, intraperitoneal, intracranial, intranasal, mucosal, anal, vaginal, oral, buccal route or they can be inhaled) or they can be administered by topical application. In some embodiments, the route of administration is intramuscular. In other embodiments, the route of administration is subcutaneous. In yet other embodiments, the route of administration is mucosal. In certain embodiments, the route of administration is transdermal or intradermal.

Certain routes of administration are particularly appropriate for vaccine formulations and immunogenic compositions comprising specified adjuvants. In particular, transdermal administration is one suitable route of administration for S. pneumoniae vaccines comprising toxins (e.g., cholera toxin or labile toxin); in other embodiments, the administration is intranasal. Vaccines formulated with Alphavirus replicons can be administered, for example, by the intramuscular or the subcutaneous route. Vaccines comprising Monophosphory Lipid A (MPL), Trehalose Dicoynomycolate (TDM), and dioctadecyldimethylammonium bromide (DDA) are suitable (inter alia) for intramuscular and subcutaneous administration. A vaccine comprising resiquimod can be administered topically or subcutaneously, for example.

3. Formulations

The vaccine formulation or immunogenic composition can be suitable for administration to a human patient, and vaccine or immunogenic composition preparation can conform to USFDA guidelines. In some embodiments, the vaccine formulation or immunogenic composition is suitable for administration to a non-human animal. In some embodiments, the vaccine or immunogenic composition is substantially free of either endotoxins or exotoxins. Endotoxins can include pyrogens, such as some lipopolysaccharide (LPS) molecules not used herein as antigens. The vaccine or immunogenic composition can also be substantially free of inactive protein fragments which may cause a fever or other side effects. In some embodiments, the composition contains less than 1%, less than 0.1%, less than 0.01%, less than 0.001%, or less than 0.0001% of endotoxins, exotoxins, and/or inactive protein fragments. In some embodiments, the vaccine or immunogenic composition has lower levels of pyrogens than industrial water, tap water, or distilled water. Other vaccine or immunogenic composition components can be purified using methods known in the art, such as ion-exchange chromatography, ultrafiltration, or distillation. In other embodiments, the pyrogens can be inactivated or destroyed prior to administration to a patient. Raw materials for vaccines, such as water, buffers, salts and other chemicals can also be screened and depyrogenated. All materials in the vaccine can be sterile, and each lot of the vaccine can be tested for sterility. Thus, in certain embodiments the endotoxin levels in the vaccine fall below the levels set by the USFDA, for example 0.2 endotoxin (EU)/kg of product for an intrathecal injectable composition; 5 EU/kg of product for a non-intrathecal injectable composition, and 0.25-0.5 EU/mL for sterile water.

In certain embodiments, the preparation comprises less than 50%, 20%, 10%, or 5% (by dry weight) contaminating protein. In certain embodiments, the desired molecule is present in the substantial absence of other biological macromolecules, such as other proteins (particularly other proteins which can substantially mask, diminish, confuse or alter the characteristics of the component proteins either as purified preparations or in their function in the subject reconstituted mixture). In certain embodiments, at least 80%, 90%, 95%, 99%, or 99.8% (by dry weight) of biological macromolecules of the same type present (but water, buffers, and other small molecules, especially molecules having a molecular weight of less than 5000, can be present). In some embodiments, the vaccine or immunogenic composition comprising purified subunit proteins contains less than 5%, 2%, 1%, 0.5%, 0.2%, 0.1% of protein from host cells in which the subunit proteins were expressed, relative to the amount of purified subunit. In some embodiments, the desired polypeptides are substantially free of nucleic acids and/or carbohydrates. For instance, in some embodiments, the vaccine or immunogenic composition contains less than 5%, less than 2%, less than 1%, less than 0.5%, less than 0.2%, or less than 0.1% host cell DNA and/or RNA. In certain embodiments, at least 80%, 90%, 95%, 99%, or 99.8% (by dry weight) of biological macromolecules of the same type are present in the preparation (but water, buffers, and other small molecules, especially molecules having a molecular weight of less than 5000, can be present).

It is preferred that the vaccine or immunogenic composition has low or no toxicity, within a reasonable risk-benefit ratio. For example, the compositions preferably have a low level of reactogenicity in animal toxicology studies. See, e.g., World Health Organization, “Procedure for assessing the acceptability, in principle, of vaccines for purchase by United Nations agencies” (WHO/IVB/05.19) published 2005; Dellepiane, N., et al., “New challenges in assuring vaccine quality,” Bulletin of the World Health Organization 78(2):155-162 (2000), which are incorporated herein by reference.

The formulations suitable for introduction of the vaccine formulations or pharmaceutical composition vary according to route of administration. Formulations suitable for parenteral administration, such as, for example, by intraarticular (in the joints), intravenous, intramuscular, intradermal, intraperitoneal, intranasal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials.

Injection solutions and suspensions can be prepared from sterile powders, granules, and tablets of the kind previously described. In the case of adoptive transfer of therapeutic T cells, the cells can be administered intravenously or parenterally.

Formulations suitable for oral administration can consist of (a) liquid solutions, such as an effective amount of the polypeptides suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. The pharmaceutical compositions can be encapsulated, e.g., in liposomes, or in a formulation that provides for slow release of the active ingredient.

The antigens, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like. Aerosol formulations can be delivered orally or nasally.

Suitable formulations for vaginal or rectal administration include, for example, suppositories, which consist of the polypeptides with a suppository base. Suitable suppository bases include natural or synthetic triglycerides or paraffin hydrocarbons. In addition, it is also possible to use gelatin rectal capsules which consist of a combination of the polypeptides with a base, including, for example, liquid triglycerides, polyethylene glycols, and paraffin hydrocarbons.

### F. Preparation and Storage

The S. pneumoniae vaccines and immunogenic compositions described herein, or the various subcomponents thereof, can be produced using a variety of techniques. For example, a polypeptide can be produced using recombinant DNA technology in a suitable host cell. A suitable host cell can be bacterial, yeast, mammalian, or other type of cell. The host cell can be modified to express an exogenous copy of one of the relevant polypeptide genes. Typically, the gene is operably linked to appropriate regulatory sequences such as a strong promoter and a polyadenylation sequence. In some embodiments, the promoter is inducible or repressible. Other regulatory sequences can provide for secretion or excretion of the polypeptide of interest or retention of the polypeptide of interest in the cytoplasm or in the membrane, depending on how one wishes to purify the polypeptide. The gene can be present on an extrachromosomal plasmid, or can be integrated into the host genome. One of skill in the art will recognize that it is not necessary to use a nucleic acid 100% identical to the naturally-occurring sequence. Rather, some alterations to these sequences are tolerated and can be desirable. For instance, the nucleic acid can be altered to take advantage of the degeneracy of the genetic code such that the encoded polypeptide remains the same. In some embodiments, the gene is codon-optimized to improve expression in a particular host. The nucleic acid can be produced, for example, by PCR or by chemical synthesis.

Once a recombinant cell line has been produced, a polypeptide can be isolated from it. The isolation can be accomplished, for example, by affinity purification techniques or by physical separation techniques (e.g., a size column).

In a further aspect of the present disclosure, there is provided a method of manufacture comprising mixing one or more polypeptides or an immunogenic fragment or variant thereof with a carrier and/or an adjuvant.

In some embodiments, antigens for inclusion the vaccine formulations and immunogenic compositions can be produced in cell culture. One method comprises providing one or more expression vectors and cloning nucleotides encoding one or more polypeptides described herein, then expressing and isolating the polypeptides.

The immunogenic polypeptides described herein and the polysaccharide antigens can be packaged in packs, dispenser devices, and kits for administering the compositions to a mammal. For example, packs or dispenser devices that contain one or more unit dosage forms are provided. Typically, instructions for administration of the compounds will be provided with the packaging, along with a suitable indication on the label that the compound is suitable for treatment of an indicated condition, such as those disclosed herein.

While various embodiments have been illustrated and described, it will be appreciated that various changes can be made therein without departing from the spirit and scope of the disclosure.

The embodiments of the invention in which an exclusive property or privilege is claimed are defined as follows:

1. A method of generating an immune response against Streptococcus pneumoniae, comprising administering to a subject in need a therapeutically effective amount of an immunogenic composition, wherein the immunogenic composition comprises:
a plurality of different S. pneumoniae capsular polysaccharides (CPs) from S. pneumoniae serotypes 1, 2, 3, 4, 5, 6B, 7F, 8, 9N, 9V, 10A, 11A, 12F, 14, 15B, 17F, 18C, 19F, 19A, 20, 22F, 23F, or 33F;
a first polypeptide antigen with an amino acid sequence at least 90% identical to the polypeptide sequence of an antigen listed in Table 2, or an immunogenic fragment thereof; or
a second polypeptide antigen with an amino acid sequence at least 90% identical to the polypeptide sequence of an antigen listed in Table 1, or an immunogenic fragment thereof;
wherein the plurality of CPs are conjugated to the first polypeptide antigen.

2. The method of claim 1, wherein the plurality of different CPs from S. pneumoniae serotypes is 1, 4, 5, 6A, 6B, 7F, 9V, 14, 18C, 19A, 19F, or 23F.

3. The method of claim 2, wherein the plurality of different CPs comprises at least one CP from each of the following serotypes 1, 4, 5, 6A, 6B, 7F, 9V, 14, 18C, 19A, 19F, and 23F.

4. The method of claim 1, wherein the first polypeptide antigen has an amino acid sequence with at least 90% identity to SEQ ID NO:22 or SEQ ID NO:36, or any immunogenic fragment thereof.

5. The method of claim 1, wherein the second polypeptide antigen has an amino acid sequence with at least 90% identity to SEQ ID NO:90, or an antigenic fragment thereof, and comprises an L(Leu)460D(Asp) substitution.

6. The method of claim 1, wherein the first and second polypeptide antigens are linked.

7. The method of claim 1, wherein the immunogenic composition further comprises aluminum-based adjuvant.

8. The method of claim 1, wherein the immunogenic compound is administered in a particle formulation.

9. The method of claim 1, wherein the administration of the immunogenic composition reduces the mucosal carriage of S. pneumoniae in the subject.

10. The method of claim 1, wherein the subject is a mammal.

11. The method of claim 1, wherein the subject is a mouse or human.


SEQUENCE LISTING (92 sequences)

SEQ ID NO: 1 - protein, 653 aa, Artificial Sequence
  Synthetic
MNKKKMILTSLASVAILGAGFVASSPTFVRAEEAPVANQSKAEKDYDAAVKKSEAAKKDYETAKKKAEDAQKKYDEDQKKTEAKAEKERKASEKIAEATKEVQQAYLAYLQASNESQRKEADKKIKEATQRKDEAEAAFATIRTTIVVPEPSELAETKKKAEEATKEAEVAKKKSEEAAKEVEVEKNKILEQDAENEKKIDVLQNKVADLEKGIAPYQNEVAELNKEIARLQSDLKDAEENNVEDYIKEGLEQAITNKKAELATTQQNIDKTQKDLEDAELELEKVLATLDPEGKTQDELDKEAAEAELNEKVEALQNQVAELEEELSKLEDNLKDAETNNVEDYIKEGLEEAIATKKAELEKTQKELDAALNELGPDGDEEETPAPAPQPEKPAEEPENPAPAPKPEKSADQQAEEDYARRSEEEYNRLTQQQPPKAEKPAPAPQPEQPAPAPKIGWKQENGMWYFYNTDGSMATGWLQNNGSWYYLNSNGAMATGWLQYNGSWYYLNANGAMATGWLQYNGSWYYLNANGAMATGWLQYNGSWYYLNANGDMATGWLQYNGSWYYLNANGDMATGWAKVHGSWYYLNANGSMATGWVKDGETWYYLEASGSMKANQWFQVSDKWYYVNGLGSLSVNTTVDGYKVNANGEWV

SEQ ID NO: 2 - protein, 283 aa, Artificial Sequence
  Synthetic
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMADLKKAVNEPEKPAEEPENPAPAPKPAPAPQPEKPAPAPAPKPEKSADQQAEEDYARRSEEEYNRLTQQQPPKAEKPAPAPVPKPEQPAPAPKTGWGQENGMWCRQACGRTRAPPPPPLRSGC

SEQ ID NO: 3 - protein, 103 aa, Artificial Sequence
  Synthetic
ADLKKAVNEPEKPAEEPENPAPAPKPAPAPQPEKPAPAPAPKPEKSADQQAEEDYARRSEEEYNRLTQQQPPKAEKPAPAPVPKPEQPAPAPKTGWGQENGMW

SEQ ID NO: 4 - protein, 58 aa, Artificial Sequence
  Synthetic
MAKKAELEKTPEKPAEEPENPAPAPQPEKSADQQAEEDYARRSEEEYNRLTQQQPPKA

SEQ ID NO: 5 - protein, 286 aa, Artificial Sequence
  Synthetic
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMADLKKAVNEPETPAPAPAPAPAPAPTPEAPAPAPAPAPKPAPAPKPAPAPKPAPAPKPAPAPKPAPAPKPAPAPAPAPKPEKPAEKPAPAPKPETPKTGWKQENGMWCRQACGRTRAPPPPPLRSG

SEQ ID NO: 6 - protein, 106 aa, Artificial Sequence
  Synthetic
DLKKAVNEPETPAPAPAPAPAPAPTPEAPAPAPAPAPKPAPAPKPAPAPKPAPAPKPAPAPKPAPAPKPAPAPAPAPKPEKPAEKPAPAPKPETPKTGWKQENGMW

SEQ ID NO: 7 - protein, 121 aa, Artificial Sequence
  Synthetic
YFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGM

SEQ ID NO: 8 - protein, 27 aa, Artificial Sequence
  Synthetic
EKSADQQAEEDYARRSEEEYNRLTQQQ

SEQ ID NO: 9 - protein, 23 aa, Artificial Sequence
  Synthetic
DQQAEEDYARRSEEEYNRLTQQQ

SEQ ID NO: 10 - protein, 28 aa, Artificial Sequence
  Synthetic
MEKSADQQAEEDYARRSEEEYNRLTQQQ

SEQ ID NO: 11 - protein, 8 aa, Artificial Sequence
  Synthetic
DLKKAVNE

SEQ ID NO: 12 - protein, 13 aa, Artificial Sequence
  Synthetic
KGTGWKGQENGMW

SEQ ID NO: 13 - protein, 165 aa, Streptococcus pneumoniae
MSYFEQFMQANQAYVALHGQLNLPLKPKTRVAIVTCMDSRLHVAQALGLALGDAHILRNAGGRVTEDMIRSLVISQQQMGTREIVVLHHTDCGAQTFENEPFQEYLKEELGVDVSDQDFLPFQDIEESVREDMQLLIESPLIPDDVIISGAIYNVDTGSMTVVEL

SEQ ID NO: 14 - protein, 274 aa, Streptococcus pneumoniae
MNQSYFYLKMKEHKLKVPYTGKERRVRILLPKDYEKDTDRSYPVVYFHDGQNVFNSKESFIGHSWKIIPAIKRNPDISRMIVVAIDNDGMGRMNEYAAWKFQESPIPGQQFGGKGVEYAEFVMEVVKPFIDETYRTKADCQHTAMIGSSLGGNITQFIGLEYQDQIGCLGVFSSANWLHQEAFNRYFECQKLSPDQRIFIYVGTEEADDTDKTLMDGNIKQAYIDSSLCYYHDLIAGGVHLDNLVLKVQSGAIHSEIPWSENLPDCLRFFAEKW

SEQ ID NO: 15 - protein, 130 aa, Streptococcus pneumoniae
MNQSYFYLKMKEHKLKVPYTGKERRVRILLPKDYEKDTDRSYPVVYFHDGQNVFNSKESFIGHSWKIIPAIKRNPDISRMIVVAIDNDGMGRMNEYAAWKFQESPIPGQQFGGKGVEYAEFVMEVVKPFI

SEQ ID NO: 16 - protein, 299 aa, Streptococcus pneumoniae
MSSKFMKSAAVLGTATLASLLLVACMNQSYFYLKMKEHKLKVPYTGKERRVRILLPKDYEKDTDRSYPVVYFHDGQNVFNSKESFIGHSWKIIPAIKRNPDISRMIVVAIDNDGMGRMNEYAAWKFQESPIPGQQFGGKGVEYAEFVMEVVKPFIDETYRTKADCQHTAMIGSSLGGNITQFIGLEYQDQIGCLGVFSSANWLHQEAFNRYFECQKLSPDQRIFIYVGTEEADDTDKTLMDGNIKQAYIDSSLCYYHDLIAGGVHLDNLVLKVQSGAIHSEIPWSENLPDCLRFFAEKW

SEQ ID NO: 17 - protein, 155 aa, Streptococcus pneumoniae
MSSKFMKSAAVLGTATLASLLLVACMNQSYFYLKMKEHKLKVPYTGKERRVRILLPKDYEKDTDRSYPVVYFHDGQNVFNSKESFIGHSWKIIPAIKRNPDISRMIVVAIDNDGMGRMNEYAAWKFQESPIPGQQFGGKGVEYAEFVMEVVKPFI

SEQ ID NO: 18 - protein, 254 aa, Streptococcus pneumoniae
MCSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIK

SEQ ID NO: 19 - protein, 276 aa, Streptococcus pneumoniae
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIK

SEQ ID NO: 20 - protein, 586 aa, Streptococcus pneumoniae
MVDKQVIEEIKNNANIVEVIGDVISLQKAGRNYLGLCPFHGEKTPSFNVVEDKQFYHCFGCGRSGDVFKFIEEYQGVPFIEAVQILGQRVGIEVEKPLYSEQKSASPHQALYDMHEDAAKFYHAILMTTTMGEEARNYLYQRGLTDEVLKHFWIGLAPPERNYLYQRLSDQYREEDLLDSGLFYLSDANQFVDTFHNRIMFPLTNDQGKVIAFSGRIWQKTDSQTSKYKNSRSTAIFNKSYELYHMDRAKRSSGKASEIYLMEGFMDVIAAYRAGIENAVASMGTALSREHVEHLKRLTKKLVLVYDGDKAGQAATLKALDEIGDMPVQIVSMPDNLDPDEYLQKNGPEDLAYLLTKTRISPIEFYIHQYKPENSENLQAQIEFLEKIAPLIVQEKSIAAQNSYIHILADSLASFDYTQIEQIVNESRQVQRQNRMEGISRPTPITMPVTKQLSAIMRAEAHLLYRMMESPLVLNDYRLREDFAFATPEFQVLYDLLGQYGNLPPEVLAEQTEEVERAWYQVLAQDLPAEISPQELSEVEMTRNKALLNQDNMRIKKKVQEASHVGDTDTALEELERLISQKRRME

SEQ ID NO: 21 - protein, 423 aa, Streptococcus pneumoniae
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGE

SEQ ID NO: 22 - protein, 400 aa, Streptococcus pneumoniae
MCGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGE

SEQ ID NO: 23 - protein, 648 aa, Streptococcus pneumoniae
MSGTSMATPIVAASTVLIRPKLKEMLERPVLKNLKGDDKIDLTSLTKIALQNTARPMMDATSWKEKSQYFASPRQQGAGLINVANALRNEVVATFKNTDSKGLVNSYGSISLKEIKGDKKYFTIKLHNTSNRPLTFKVSASAITTDSLTDRLKLDETYKDEKSPDGKQIVPEIHPEKVKGANITFEHDTFTIGANSSFDLNAVINVGEAKNKNKFVESFIHFESVEEMEALNSNGKKINFQPSLSMPLMGFAGNWNHEPILDKWAWEEGSRSKTLGGYDDDGKPKIPGTLNKGIGGEHGIDKFNPAGVIQNRKDKNTTSLDQNPELFAFNNEGINAPSSSGSKIANIYPLDSNGNPQDAQLERGLTPSPLVLRSAEEGLISIVNTNKEGENQRDLKVISREHFIRGILNSKSNDAKGIKSSKLKVWGDLKWDGLIYNPRGREENAPESKDNQDPATKIRGQFEPIAEGQYFYKFKYRLTKDYPWQVSYIPVKIDNTAPKIVSVDFSNPEKIKLITKDTYHKVKDQYKNETLFARDQKEHPEKFDEIANEVWYAGAALVNEDGEVEKNLEVTYAGEGQGRNRKLDKDGNTIYEIKGAGDLRGKIIEVIALDGSSNFTKIHRIKFANQADEKGMISYYLVDPDQDSSKYQ

SEQ ID NO: 24 - protein, 2140 aa, Streptococcus pneumoniae
MKKSTVLSLTTAAVILAAYAPNEVVLADTSSSEDALNISDKEKVAENKEKHENIHSAMETSQDFKEKKTAVIKEKEVVSKNPVIDNNTSNEEAKIKEENSNKSQGDYTDSFVNKNTENPKKEDKVVYIAEFKDKESGEKAIKELSSLKNTKVLYTYDRIFNGSAIETTPDNLDKIKQIEGISSVERAQKVQPMMNHARKEIGVEEAIDYLKSINAPFGKNFDGRGMVISNIDTGTDYRHKAMRIDDDAKASMRFKKEDLKGTDKNYWLSDKIPHAFNYYNGGKITVEKYDDGRDYFDPHGMHIAGILAGNDTEQDIKNFNGIDGIAPNAQIFSYKMYSDAGSGFAGDETMFHAIEDSIKHNVDVVSVSSGFTGTGLVGEKYWQAIRALRKAGIPMVVATGNYATSASSSSWDLVANNHLKMTDTGNVTRTAAHEDAIAVASAKNQTVEFDKVNIGGESFKYRNIGAFFDKSKITTNEDGTKAPSKLKFVYIGKGQDQDLIGLDLRGKIAVMDRIYTKDLKNAFKKAMDKGARAIMVVNTVNYYNRDNWTELPAMGYEADEGTKSQVFSISGDDGVKLWNMINPDKKTEVKRNNKEDFKDKLEQYYPIDMESFNSNKPNVGDEKEIDFKFAPDTDKELYKEDIIVPAGSTSWGPRIDLLLKPDVSAPGKNIKSTLNVINGKSTYGYMSGTSMATPIVAASTVLIRPKLKEMLERPVLKNLKGDDKIDLTSLTKIALQNTARPMMDATSWKEKSQYFASPRQQGAGLINVANALRNEVVATFKNTDSKGLVNSYGSISLKEIKGDKKYFTIKLHNTSNRPLTFKVSASAITTDSLTDRLKLDETYKDEKSPDGKQIVPEIHPEKVKGANITFEHDTFTIGANSSFDLNAVINVGEAKNKNKFVESFIHFESVEEMEALNSNGKKINFQPSLSMPLMGFAGNWNHEPILDKWAWEEGSRSKTLGGYDDDGKPKIPGTLNKGIGGEHGIDKFNPAGVIQNRKDKNTTSLDQNPELFAFNNEGINAPSSSGSKIANIYPLDSNGNPQDAQLERGLTPSPLVLRSAEEGLISIVNTNKEGENQRDLKVISREHFIRGILNSKSNDAKGIKSSKLKVWGDLKWDGLIYNPRGREENAPESKDNQDPATKIRGQFEPIAEGQYFYKFKYRLTKDYPWQVSYIPVKIDNTAPKIVSVDFSNPEKIKLITKDTYHKVKDQYKNETLFARDQKEHPEKFDEIANEVWYAGAALVNEDGEVEKNLEVTYAGEGQGRNRKLDKDGNTIYEIKGAGDLRGKIIEVIALDGSSNFTKIHRIKFANQADEKGMISYYLVDPDQDSSKYQKLGEIAESKFKNLGNGKEGSLKKDTTGVEHHHQENEESIKEKSSFTIDRNISTIRDFENKDLKKLIKKKFREVDDFTSETGKRMEEYDYKYDDKGNIIAYDDGTDLEYETEKLDEIKSKIYGVLSPSKDGHFEILGKISNVSKNAKVYYGNNYKSIEIKATKYDFHSKTMTFDLYANINDIVDGLAFAGDMRLFVKDNDQKKAEIKIRMPEKIKETKSEYPYVSSYGNVIELGEGDLSKNKPDNLTKMESGKIYSDSEKQQYLLKDNIILRKGYALKVTTYNPGKTDMLEGNGVYSKEDIAKIQKANPNLRALSETTIYADSRNVEDGRSTQSVLMSALDGFNIIRYQVFTFKMNDKGEAIDKDGNLVTDSSKLVLFGKDDKEYTGEDKFNVEAIKEDGSMLFIDTKPVNLSMDKNYFNPSKSNKIYVRNPEFYLRGKISDKGGFNWELRVNESVVDNYLIYGDLHIDNTRDFNIKLNVKDGDIMDWGMKDYKANGFPDKVTDMDGNVYLQTGYSDLNAKAVGVHYQFLYDNVKPEVNIDPKGNTSIEYADGKSVVFNINDKRNNGFDGEIQEQHIYINGKEYTSFNDIKQIIDKTLNIKIVVKDFARNTTVKEFILNKDTGEVSELKPHRVTVTIQNGKEMSSTIVSEEDFILPVYKGELEKGYQFDGWEISGFEGKKDAGYVINLSKDTFIKPVFKKIEEKKEEENKPTFDVSKKKDNPQVNHSQLNESHRKEDLQREEHSQKSDSTKDVTATVLDKNNISSKSTTNNPNKLPKTGTASGAQTLLAAGIMFIVGIFLGLKKKNQD

SEQ ID NO: 25 - protein, 662 aa, Streptococcus pneumoniae
MVVLADTSSSEDALNISDKEKVAENKEKHENIHSAMETSQDFKEKKTAVIKEKEVVSKNPVIDNNTSNEEAKIKEENSNKSQGDYTDSFVNKNTENPKKEDKVVYIAEFKDKESGEKAIKELSSLKNTKVLYTYDRIFNGSAIETTPDNLDKIKQIEGISSVERAQKVQPMMNHARKEIGVEEAIDYLKSINAPFGKNFDGRGMVISNIDTGTDYRHKAMRIDDDAKASMRFKKEDLKGTDKNYWLSDKIPHAFNYYNGGKITVEKYDDGRDYFDPHGMHIAGILAGNDTEQDIKNFNGIDGIAPNAQIFSYKMYSDAGSGFAGDETMFHAIEDSIKHNVDVVSVSSGFTGTGLVGEKYWQAIRALRKAGIPMVVATGNYATSASSSSWDLVANNHLKMTDTGNVTRTAAHEDAIAVASAKNQTVEFDKVNIGGESFKYRNIGAFFDKSKITTNEDGTKAPSKLKFVYIGKGQDQDLIGLDLRGKIAVMDRIYTKDLKNAFKKAMDKGARAIMVVNTVNYYNRDNWTELPAMGYEADEGTKSQVFSISGDDGVKLWNMINPDKKTEVKRNNKEDFKDKLEQYYPIDMESFNSNKPNVGDEKEIDFKFAPDTDKELYKEDIIVPAGSTSWGPRIDLLLKPDVSAPGKNIKSTLNVINGKSTYG

SEQ ID NO: 26 - protein, 274 aa, Artificial Sequence
  Synthetic
XNQSYFYLKMKEHKLKVPYTGKERRVRILLPKDYEKDTDRSYPVVYFHDGQNVFXSKESFIGXSWKIIPAIKRNPDISXMIVVAIDNDGMGRMNEYXAWKFQESPIPXQQFGGKGVEYAEFVMEVVKPFIDETYRTKADCQHTAMIGSSLGGNITQFIGLEYQXXIGCLGVFSSANWLHQEAFNRYXECQKLSPDQXIFIYVGTEEADDTDKTLMDGNIKQAYIDSSLCYYHDLIAGXVHLDNLVLKVQSGAIHSEIPWSENLPDCLRFFAEKW

SEQ ID NO: 27 - protein, 130 aa, Artificial Sequence
  Synthetic
XNQSYFYLKMKEHKLKVPYTGKERRVRILLPKDYEKDTDRSYPVVYFHDGQNVFXSKESFIGXSWKIIPAIKRNPDISXMIVVAIDNDGMGRMNEYXAWKFQESPIPXQQFGGKGVEYAEFVMEVVKPFI

SEQ ID NO: 28 - protein, 299 aa, Artificial Sequence
  Synthetic
MSSKFXKSXAVLGTXTLASLLLVACXNQSYFYLKMKEHKLKVPYTGKERRVRILLPKDYEKDTDRSYPVVYFHDGQNVFXSKESFIGXSWKIIPAIKRNPDISXMIVVAIDNDGMGRMNEYXAWKFQESPIPXQQFGGKGVEYAEFVMEVVKPFIDETYRTKADCQHTAMIGSSLGGNITQFIGLEYQXXIGCLGVFSSANWLHQEAFNRYXECQKLSPDQXIFIYVGTEEADDTDKTLMDGNIKQAYIDSSLCYYHDLIAGXVHLDNLVLKVQSGAIHSEIPWSENLPDCLRFFAEKW

SEQ ID NO: 29 - protein, 155 aa, Artificial Sequence
  Synthetic
MSSKFXKSXAVLGTXTLASLLLVACXNQSYFYLKMKEHKLKVPYTGKERRVRILLPKDYEKDTDRSYPVVYFHDGQNVFXSKESFIGXSWKIIPAIKRNPDISXMIVVAIDNDGMGRMNEYXAWKFQESPIPXQQFGGKGVEYAEFVMEVVKPFI

SEQ ID NO: 30 - protein, 254 aa, Artificial Sequence
  Synthetic
MCSGGAKKEGXAASKKEIIVATNXSPXPFXYEENGELTGYEIEVVRAIFKDSDKYXVXFEKTEWSGVFAGLDADRYNMAVNNXSYTKERAEKYLYAAPIAQNPNVLVVKKXDSSIKSLDDIGGKSTEVVQATTSAKQLEAYNAEHTDNPTILNYTKADXQQIMVRLSDGQFDYKIFDKIGVETVIKNQGLDXLKVIELXSDQQPYVYPLLAQGQDELKSFVDKRIKELYKDGTLEKLSKQFFGDTYLPAEADIK

SEQ ID NO: 31 - protein, 276 aa, Artificial Sequence
  Synthetic
MKKIVKYSSLAALXLVAAGXLAACSGGAKKEGXAASKKEIIVATNXSPXPFXYEENGELTGYEIEVVRAIFKDSDKYXVXFEKTEWSGVFAGLDADRYNMAVNNXSYTKERAEKYLYAAPIAQNPNVLVVKKXDSSIKSLDDIGGKSTEVVQATTSAKQLEAYNAEHTDNPTILNYTKADXQQIMVRLSDGQFDYKIFDKIGVETVIKNQGLDXLKVIELXSDQQPYVYPLLAQGQDELKSFVDKRIKELYKDGTLEKLSKQFFGDTYLPAEADIK

SEQ ID NO: 32 - protein, 400 aa, Artificial Sequence
  Synthetic
MCGSKTADKPADSGSSEXKELTVYVDEGYKSYIEEVAKAYEKEAGVKXTLKTGDALGGLDKLSLDNQSGNVPDXMMAPYDRVXSLGSDGQLSEVKLSDGXKTDDTTKSLVTAANGKVYGAPAVIESLVMYYNKDLVKDAPKTFADLENLAKDSKYAFAGEDGKTTAFLADWTNFYYXYGLLAGNGXYVFGQNGKDXKDIGLANDGSIXGINYAXSWYEKWPKGMQDTEGAGNLIQTXFQEGKTAAIIDGPWKAQAFKDAKVNYGVATIPTLPNGKEYAAFGGGKAWVIPQAVKNLEAXQKFVDFLVXTEQQKXLYDKTNEIPANTEARSYAEGKNDELTTAVIKQFKXTQPLPNISQMSAVWDPAKNMLFDAVSGQKDAKTAANDAVTLIKETIKQKFGE

SEQ ID NO: 33 - protein, 423 aa, Artificial Sequence
  Synthetic
MSSKFXKSXAVLGTXTLASLLLVACGSKTADKPADSGSSEXKELTVYVDEGYKSYIEEVAKAYEKEAGVKXTLKTGDALGGLDKLSLDNQSGNVPDXMMAPYDRVXSLGSDGQLSEVKLSDGXKTDDTTKSLVTAANGKVYGAPAVIESLVMYYNKDLVKDAPKTFADLENLAKDSKYAFAGEDGKTTAFLADWTNFYYXYGLLAGNGXYVFGQNGKDXKDIGLANDGSIXGINYAXSWYEKWPKGMQDTEGAGNLIQTXFQEGKTAAIIDGPWKAQAFKDAKVNYGVATIPTLPNGKEYAAFGGGKAWVIPQAVKNLEAXQKFVDFLVXTEQQKXLYDKTNEIPANTEARSYAEGKNDELTTAVIKQFKXTQPLPNISQMSAVWDPAKNMLFDAVSGQKDAKTAANDAVTLIKETIKQKFGE

SEQ ID NO: 34 - protein, 357 aa, Artificial Sequence
  Synthetic
MANIFDYLKDVAYDSYYDLPLNELDILTLIEITYLSFDNLVSTLPQRLLDLAPQVPRDPTMLTSKNRLQLLDELAQHKRFKNCKLSHFINDIDPELQKQFAAMTYRVSLDTYLIVFRGTDDSIIGWKEDFHLTYMKEIPAQKHALRYLKNFFAHHPKQKVILAGHSKGGNLAIYAASQIEQSLQNQITAVYTFDAPGLHQELTQTAGYQRIMDRSKIFIPQGSIIGMMLEIPAHQIIVQSTALGGIAQHDTFSWQIEDKHFVQLDKTNSDSQQVDTTFKEWVATVPDEELQLYFDLFFGTILDAGISSINDLASLKALEYIHHLFVQAQSLTPEERETLGRLTQLLIDTRYQAWKNR

SEQ ID NO: 35 - protein, 1066 aa, Streptococcus pneumoniae
MQTKTKKLIVSLSSLVLSGFLLNHYMTIGAEETTTNTIQQSQKEVQYQQRDTKNLVENGDFGQTEDGSSPWTGSKAQGWSAWVDQKNSADASTRVIEAKDGAITISSHEKLRAALHRMVPIEAKKKYKLRFKIKTDNKIGIAKVRIIEESGKDKRLWNSATTSGTKDWQTIEADYSPTLDVDKIKLELFYETGTGTVSFKDIELVEVADQLSEDSQTDKQLEEKIDLPIGKKHVFSLADYTYKVENPDVASVKNGILEPLKEGTTNVIVSKDGKEVKKIPLKILASVKDAYTDRLDDWNGIIAGNQYYDSKNEQMAKLNQELEGKVADSLSSISSQADRTYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQCLQSLVKTIVQSDSYYDVFKNLKTYKDISLMQSLLSDAGVASVPRPSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESGNPYKVYVNDKEASLTEQEKDYPETQSVFLESFDSKKNIGYFFFKKSSISMSKALQKGAWKDINEGQSDKEVENEFLTISQAHKQNRDSYGYMLIPNVDRATFNQMIKELESSLIENNETLQSVYDAKQGVWGIVKYDDSVSTISNQFQVLKRGVYTIRKEGDEYKIAYYNPETQESAPDQEVFKKLEQAAQPQVQNSKEKEKSEEEKNHSDQKNLPQTGEGQSILASLGFLLLGAFYLFRRGKNN

SEQ ID NO: 36 - protein, 99 aa, Streptococcus pneumoniae
MNGMKAKKMWMAGLALLGIGSLALATKKVADDRKLMKTQEELTEIVRDHFSDMGEIATLYVQVYESSLESLVGGVIFEDGRHYTFVYENEDLVYEEEVL

SEQ ID NO: 37 - protein, 92 aa, Streptococcus pneumoniae
MRYLATLLLSLAVLITAGCKKVADDRKLMKTQEELTEIVRDHFSDMGEIATLYVQVYESSLESLVGGVIFEDGRHYTFVYENEDLVYEEEVL

SEQ ID NO: 38 - protein, 978 aa, Streptococcus pneumoniae
DTSSSEDALNISDKEKVAENKEKHENIHSAMETSQDFKEKKTAVIKEKEVVSKNPVIDNNTSNEEAKIKEENSNKSQGDYTDSFVNKNTENPKKEDKVVYIAEFKDKESGEKAIKELSSLKNTKVLYTYDRIFNGSAIETTPDNLDKIKQIEGISSVERAQKVQPMMNHARKEIGVEEAIDYLKSINAPFGKNFDGRGMVISNIDTGTDYRHKAMRIDDDAKASMRFKKEDLKGTDKNYWLSDKIPHAFNYYNGGKITVEKYDDGRDYFDPHGMHIAGILAGNDTEQDIKNFNGIDGIAPNAQIFSYKMYSDAGSGFAGDETMFHAIEDSIKHNVDVVSVSSGFTGTGLVGEKYWQAIRALRKAGIPMVVATGNYATSASSSSWDLVANNHLKMTDTGNVTRTAAHEDAIAVASAKNQTVEFDKVNIGGESFKYRNIGAFFDKSKITTNEDGTKAPSKLKFVYIGKGQDQDLIGLDLRGKIAVMDRIYTKDLKNAFKKAMDKGARAIMVVNTVNYYNRDNWTELPAMGYEADEGTKSQVFSISGDDGVKLWNMINPDKKTEVKRNNKEDFKDKLEQYYPIDMESFNSNKPNVGDEKEIDFKFAPDTDKELYKEDIIVPAGSTSWGPRIDLLLKPDVSAPGKNIKSTLNVINGKSTYGYMSGTSMATPIVAASTVLIRPKLKEMLERPVLKNLKGDDKIDLTSLTKIALQNTARPMMDATSWKEKSQYFASPRQQGAGLINVANALRNEVVATFKNTDSKGLVNSYGSISLKEIKGDKKYFTIKLHNTSNRPLTFKVSASAITTDSLTDRLKLDETYKDEKSPDGKQIVPEIHPEKVKGANITFEHDTFTIGANSSFDLNAVINVGEAKNKNKFVESFIHFESVEEMEALNSNGKKINFQPSLSMPLMGFAGNWNHEPILDKWAWEEGSRSKTLGGYDDDGKPKIPGTLNKGIGGEHGIDKFNPAGVIQNRKDKNTTSL

SEQ ID NO: 39 - protein, 99 aa, Artificial Sequence
  Synthetic
MNGMKAKKMWMAGLALLGIGSLALATKKVADDXKLMKTQEELTXIVRDXFSDMGEIATLYXQVYESSLESLXGXVIFEDGRHYTFXYENEDLVYEEEVL

SEQ ID NO: 40 - protein, 667 aa, Artificial Sequence
  Synthetic
XVVLADTSXSEDALXISDKEKVXXXXXXENKEKHXXIHXAXETSXDXXEKKTXXIXXKXVVSKNPVXDXXTSNEEAXIKEXXSNXSQGDXXXSXXNKXTENPKKXDXXVYIAEFKDKESGXKAIKXLSXLKNTKVLYTYDRIFNGXAIETTXDNLDKIKQIEGIXSXERAQKVQPMMNHARKEIGVEEAIDYLKSINAPFGKNFDGRGMVISNIDTGTDYRHKAMRIDDDAKASMRFKKEDLKGTDKNYWLSDKIPHAFNYYNGGKITVEKYDDGRDYFDPHGMHIAGILAGNDTEQDIKNFNGIDGIAPNAQIFSYKMYSDAGSGFAGDETMFHAIEDSIKHNVDVVSVSSGFTGTGLVGEKYWQAIRALRKAGIPMVVATGNYATSASSSSWDLVANNHLKMTDTGNVTRTAAHEDAIAVASAKNQTVEFDKVNIGGXSFKYRNIGAFFDKXKITTNEDGTKAPSKLKFVYIGKGQDQDLIGLDLRGKIAVMDRIYTKDLKNAFKKAMDKGARAIMVVNTVNYYNRDNWTELPAMGYEADEGTKSQVFSISGDDGVKLWNMINPXKKTEVKRNNKEDFKDKLEQYYPIDMESFNSNKPNVGDEKEIDFKFAPDTDKELYKEDIIVPAGSTSWGPRIDLLLKPDVSAPGKNIKSTLNVINGKSTYG

SEQ ID NO: 41 - protein, 621 aa, Artificial Sequence
  Synthetic
MSGTSMATPIVAASTVLIRPKLKEMLEXPVLKNLXGDDKIDLTSLTKIALQNTARPMMDATSWKEKSQYFASPRQQGAGLINVANALRNEVVATFKNTDSKGLVNSYGSISLKEIKGDKKYFTIKLHNTSNRPLTFKVSASAXTTDSLTDRLKLDETYKDEKSPXGKQIVPEIHPEKVKGANITFEHDTFTIGANSSFDLNAVINVGEAXNKNKFVESFIHFESVEXMEALXSNGKKXXFQPSLSMPLMGFAGNWNHEPILDKWAWEEGSXSKTXXGYDDDGKPKIPGTLNKGIGGEHGIDKFNPAGVIQNRKDKNXTSLDQXPXLFAFNNXGXXAXSXSGSKIANIYPLDSNGNPQDAQLERGLTPSPLVLRSAEEGLISIVNTNKEGENQXDLKVXSREHFIRGILNSKXNDAKGIKSSKLKVWGDLKWDGLIYNPRGREENAPESKDNQDPATKIRGQFEPIAEGQYFYKFKYRLTKDYPWQVSYIPVKIDNTAPKIVSVDFSNPEKIKLITKDTYHKVKDQYKNETLFARDQKEHPEKFDEIANEVWYAGAALVNEDGEVEKNLEVTYAGEGQGRNRKLDKDGNTIYEIXGAGDLRGKIIEVIALDGXSNFTDHKAE

SEQ ID NO: 42 - protein, 19 aa, Artificial Sequence
  Synthetic
LGGGGSGGGGSGGGGSAAA

SEQ ID NO: 43 - protein, 20 aa, Artificial Sequence
  Synthetic
LAEATAKEATAKEATAKATA

SEQ ID NO: 44 - protein, 14 aa, Artificial Sequence
  Synthetic
GPKPHRIQSTPKGS

SEQ ID NO: 45 - DNA, 2025 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaatcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaa

SEQ ID NO: 46 - protein, 675 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGESGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIK

SEQ ID NO: 47 - DNA, 2022 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttscaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaa

SEQ ID NO: 48 - protein, 674 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGE

SEQ ID NO: 49 - DNA, 1563 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaaaatggtatgaaagctaaaaaaatgtggatggcaggcttggctctgctaggtatcggaagccttgctcttgctacgaaaaaagttgcagatgaccgtaagctcatgaagactcaggaagagttgacagagattgtgcgagaccatttttccgacatgggggaaattgcgaccctttatgttcaagtttacgaaagcagtctggagagcttggttggtggcgtcatttttgaggatggccgtcattatacctttgtctatgaaaatgaagacctagtctatgaggaggaagtctta

SEQ ID NO: 50 - protein, 521 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGENGMKAKKMWMAGLALLGIGSLALATKKVADDRKLMKTQEELTEIVRDHFSDMGEIATLYVQVYESSLESLVGGVIFEDGRHYTFVYENEDLVYEEEVL

SEQ ID NO: 51 - DNA, 1122 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaaatggtatgaaagctaaaaaaatgtggatggcaggcttggctctgctaggtatcggaagccttgctcttgctacgaaaaaagttgcagatgaccgtaagctcatgaagactcaggaagagttgacagagattgtgcgagaccatttttccgacatgggggaaattgcgaccctttatgttcaagtttacgaaagcagtctggagagcttggttggtggcgtcatttttgaggatggccgtcattatacctttgtctatgaaaatgaagacctagtctatgaggaggaagtctta

SEQ ID NO: 52 - protein, 374 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKNGMKAKKMWMAGLALLGIGSLALATKKVADDRKLMKTQEELTEIVRDHFSDMGEIATLYVQVYESSLESLVGGVIFEDGRHYTFVYENEDLVYEEEVL

SEQ ID NO: 53 - DNA, 2322 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaaaatggtatgaaagctaaaaaaatgtggatggcaggcttggctctgctaggtatcggaagccttgctcttgctacgaaaaaagttgcagatgaccgtaagctcatgaagactcaggaagagttgacagagattgtgcgagaccatttttccgacatgggggaaattgcgaccctttatgttcaagtttacgaaagcagtctggagagcttggttggtggcgtcatttttgaggatggccgtcattatacctttgtctatgaaaatgaagacctagtctatgaggaggaagtcttatgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaa

SEQ ID NO: 54 - protein, 773 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGENGMKAKKMWMAGLALLGIGSLALATKKVADDRKLMKTQEELTEIVRDHFSDMGEIATLYVQVYESSLESLVGGVIFEDGRHYTFVYENEDLVYEEEVLSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIK

SEQ ID NO: 55 - DNA, 2337 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaaatggtatgaaagctaaaaaaatgtggatggcaggcttggctctgctaggtatcggaagccttgctcttgctacgaaaaaagttgcagatgaccgtaagctcatgaagactcaggaagagttgacagagattgtgcgagaccatttttccgacatgggggaaattgcgaccctttatgttcaagtttacgaaagcagtctggagagcttggttggtggcgtcatttttgaggatggccgtcattatacctttgtctatgaaaatgaagacctagtctatgaggaggaagtcttaggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaacaccaccaccaccaccactga

SEQ ID NO: 56 - protein, 772 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKNGMKAKKMWMAGLALLGIGSLALATKKVADDRKLMKTQEELTEIVRDHFSDMGEIATLYVQVYESSLESLVGGVIFEDGRHYTFVYENEDLVYEEEVLGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGE

SEQ ID NO: 57 - DNA, 2319 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaatcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaaatggtatgaaagctaaaaaaatgtggatggcaggcttggctctgctaggtatcggaagccttgctcttgctacgaaaaaagttgcagatgaccgtaagctcatgaagactcaggaagagttgacagagattgtgcgagaccatttttccgacatgggggaaattgcgaccctttatgttcaagtttacgaaagcagtctggagagcttggttggtggcgtcatttttgaggatggccgtcattatacctttgtctatgaaaatgaagacctagtctatgaggaggaagtctta

SEQ ID NO: 58 - protein, 773 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGESGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKNGMKAKKMWMAGLALLGIGSLALATKKVADDRKLMKTQEELTEIVRDHFSDMGEIATLYVQVYESSLESLVGGVIFEDGRHYTFVYENEDLVYEEEVL

SEQ ID NO: 59 - DNA, 2316 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaaaatggtatgaaagctaaaaaaatgtggatggcaggcttggctctgctaggtatcggaagccttgctcttgctacgaaaaaagttgcagatgaccgtaagctcatgaagactcaggaagagttgacagagattgtgcgagaccatttttccgacatgggggaaattgcgaccctttatgttcaagtttacgaaagcagtctggagagcttggttggtggcgtcatttttgaggatggccgtcattatacctttgtctatgaaaatgaagacctagtctatgaggaggaagtctta

SEQ ID NO: 60 - protein, 772 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGENGMKAKKMWMAGLALLGIGSLALATKKVADDRKLMKTQEELTEIVRDHFSDMGEIATLYVQVYESSLESLVGGVIFEDGRHYTFVYENEDLVYEEEVL

SEQ ID NO: 61 - DNA, 1170 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaatggccatggccgacctgaagaaggccgtgaacgagccggagaaaccggccgaagaggagccggagaacccggcaccggcacctaaacctgcacctgcaccgcagcctgagaagcctgcccctgcaccggcaccgaaaccggagaagagcgccgaccagcaggcagaggaagactacgcccgccgcagcgaagaggagtacaatcgcctgacccagcagcaaccgccgaaggcagaaaagccggccccggccccggttccgaaacctgaacagccggcacctgcacctaaaacaggctggaagcaggaaaacggcatgtggcaccaccaccaccaccactga

SEQ ID NO: 62 - protein, 389 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKMAMADLKKAVNEPEKPAEEEPENPAPAPKPAPAPQPEKPAPAPAPKPEKSADQQAEEDYARRSEEEYNRLTQQQPPKAEKPAPAPVPKPEQPAPAPKTGWKQENGMWHHHHHH

SEQ ID NO: 63 - DNA, 1215 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaatacttcaaagagggcttagagaagaccatcgccgccaagaaggccgagttagagaaaaccgaggccgacctgaagaaagccgtgaacgagccggagaagcctgcacctgcacctgaaacacctgcccctgaagcaccggccgaacagcctaagccggcaccggcacctcaacctgccccggcaccgaagccggaaaaaccggccgagcagccgaaaccggagaaaaccgacgaccagcaggcagaagaggactatgcccgccgcagcgaggaagagtacaaccgcctgacccagcagcagcctccgaaagccgagaaaccggccccggccccgaaaaccggctggaagcaggagaacggcatgtggcaccaccaccaccaccactga

SEQ ID NO: 64 - protein, 404 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWHHHHHH

SEQ ID NO: 65 - DNA, 1611 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaaatggccatggccgacctgaagaaggccgtgaacgagccggagaaaccggccgaagaggagccggagaacccggcaccggcacctaaacctgcacctgcaccgcagcctgagaagcctgcccctgcaccggcaccgaaaccggagaagagcgccgaccagcaggcagaggaagactacgcccgccgcagcgaagaggagtacaatcgcctgacccagcagcaaccgccgaaggcagaaaagccggccccggccccggttccgaaacctgaacagccggcacctgcacctaaaacaggctggaagcaggaaaacggcatgtggcaccaccaccaccaccactga

SEQ ID NO: 66 - protein, 536 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGEMAMADLKKAVNEPEKPAEEEPENPAPAPKPAPAPQPEKPAPAPAPKPEKSADQQAEEDYARRSEEEYNRLTQQQPPKAEKPAPAPVPKPEQPAPAPKTGWKQENGMWHHHHHH

SEQ ID NO: 67 - DNA, 1656 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaatacttcaaagagggcttagagaagaccatcgccgccaagaaggccgagttagagaaaaccgaggccgacctgaagaaagccgtgaacgagccggagaagcctgcacctgcacctgaaacacctgcccctgaagcaccggccgaacagcctaagccggcaccggcacctcaacctgccccggcaccgaagccggaaaaaccggccgagcagccgaaaccggagaaaaccgacgaccagcaggcagaagaggactatgcccgccgcagcgaggaagagtacaaccgcctgacccagcagcagcctccgaaagccgagaaaccggccccggccccgaaaaccggctggaagcaggagaacggcatgtggcaccaccaccaccaccactga

SEQ ID NO: 68 - protein, 551 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGEYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWHHHHHH

SEQ ID NO: 69 - DNA, 1212 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaggcccgaagccgcaccgtatccagagcaccccgaagggcagcatggccatggcagacctgaagaaggccgtgaacgagccggaaaaacctgccgaagaggagccggaaaatcctgcccctgcacctaaacctgcacctgcccctcagccggagaaaccggcacctgcaccggcacctaagccggagaagagcgccgatcagcaggccgaggaggactatgcccgtcgcagcgaagaggagtacaaccgcctgacccagcaacagcctccgaaggccgaaaaaccggcccctgcaccggtgcctaagcctgagcaacctgccccggccccgaaaaccggctggaagcaggaaaacggcatgtggcaccaccaccaccaccactga

SEQ ID NO: 70 - protein, 403 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKGPKPHRIQSTPKGSMAMADLKKAVNEPEKPAEEEPENPAPAPKPAPAPQPEKPAPAPAPKPEKSADQQAEEDYARRSEEEYNRLTQQQPPKAEKPAPAPVPKPEQPAPAPKTGWKQENGMWHHHHHH

SEQ ID NO: 71 - DNA, 1257 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaaggcccgaagccgcaccgyatccagagcacaccgaaaggcagctacttcaaagagggcttagagaagaccatcgccgccaagaaggccgagttagagaaaaccgaggccgacctgaagaaggccgtgaacgagccggagaaacctgcacctgcaccggagacaccggcacctgaagcccctgcagagcagcctaaacctgcccctgcacctcaaccggcacctgcacctaagccggaaaaacctgccgagcagccgaagccggagaaaaccgacgaccagcaggccgaagaggactatgcacgccgcagcgaagaggagtacaaccgcttaacccaacagcagccgccgaaggcagaaaaaccggcccctgcccctaagaccggttggaagcaggagaacggcatgtggcaccaccaccaccaccactga

SEQ ID NO: 72 - protein, 418 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKGPKPHRIQSTPKGSYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWHHHHHH

SEQ ID NO: 73 - DNA, 1653 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaaggcccgaagccgcaccgtatccagagcaccccgaagggcagcatggccatggcagacctgaagaaggccgtgaacgagccggaaaaacctgccgaagaggagccggaaaatcctgcccctgcacctaaacctgcacctgcccctcagccggagaaaccggcacctgcaccggcacctaagccggagaagagcgccgatcagcaggccgaggaggactatgcccgtcgcagcgaagaggagtacaaccgcctgacccagcaacagcctccgaaggccgaaaaaccggcccctgcaccggtgcctaagcctgagcaacctgccccggccccgaaaaccggctggaagcaggagaatggcatgtggcaccaccaccaccaccactga

SEQ ID NO: 74 - protein, 550 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGEGPKPHRIQSTPKGSMAMADLKKAVNEPEKPAEEEPENPAPAPKPAPAPQPEKPAPAPAPKPEKSADQQAEEDYARRSEEEYNRLTQQQPPKAEKPAPAPVPKPEQPAPAPKTGWKQENGMWHHHHHH

SEQ ID NO: 75 - DNA, 1698 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaaggcccgaagccgcaccgcatccagagcacaccgaaaggcagctacttcaaagagggcttagagaagaccatcgccgccaagaaggccgagttagagaaaaccgaggccgacctgaagaaggccgtgaacgagccggagaaacctgcacctgcaccggagacaccggcacctgaagcccctgcagagcagcctaaacctgcccctgcacctcaaccggcacctgcacctaagccggaaaaacctgccgagcagccgaagccggagaaaaccgacgaccagcaggccgaagaggactatgcacgccgcagcgaagaggagtacaaccgcttaacccaacagcagccgccgaaggcagaaaaaccggcccctgcccctaagaccggttggaagcaggagaacggcatgtggcaccaccaccaccaccactga

SEQ ID NO: 76 - protein, 565 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGEGPKPHRIQSTPKGSYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWHHHHHH

SEQ ID NO: 77 - DNA, 1230 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaactggccgaagcaaccgccaaagaagccaccgccaaggaagccacagccaaggccaccgcaatggccatggccgacctgaagaaggccgtgaatgagccggagaagccggccgaagaagagccggagaatcctgcaccggcccctaagcctgcaccggcacctcagcctgaaaaaccggcacctgcacctgccccgaagcctgaaaagagcgccgaccagcaggccgaagaggactatgcccgccgcagcgaagaggagtacaaccgtctgacccagcagcagccgccgaaagccgagaaacctgccccggcaccggtgcctaagccggaacagcctgcccctgcaccgaagaccggttggaagcaggaaaacggcatgtggcaccaccaccaccaccactga

SEQ ID NO: 78 - protein, 409 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKLAEATAKEATAKEATAKATAMAMADLKKAVNEPEKPAEEEPENPAPAPKPAPAPQPEKPAPAPAPKPEKSADQQAEEDYARRSEEEYNRLTQQQPPKAEKPAPAPVPKPEQPAPAPKTGWKQENGMWHHHHHH

SEQ ID NO: 79 - DNA, 1275 bp, Artificial Sequence
  Synthetic
atgaaaaaaatcgttaaatactcatctcttgcagcccttgctcttgttgctgcaggtgtgcttgcggcttgctcagggggtgctaagaaagaaggagaagcagctagcaagaaagaaatcatcgttgcaaccaatggatcaccaaagccatttatctatgaagaaaatggcgaattgactggttacgagattgaagtcgttcgcgctatctttaaagattctgacaaatatgatgtcaagtttgaaaagacagaatggtcaggtgtctttgctggtcttgacgctgatcgttacaatatggctgtcaacaatcttagctacactaaagaacgtgcggagaaatacctctatgccgcaccaattgcccaaaatcctaatgtccttgtcgtgaagaaagatgactctagtatcaagtctctcgatgatatcggtggaaaatcgacggaagtcgttcaagccactacatcagctaagcagttagaagcatacaatgctgaacacacggacaacccaactatccttaactatactaaggcagacttccaacaaatcatggtacgtttgagcgatggacaatttgactataagatttttgataaaatcggtgttgaaacagtgatcaagaaccaaggtttggacaacttgaaagttatcgaacttccaagcgaccaacaaccgtacgtttacccacttcttgctcagggtcaagatgagttgaaatcgtttgtagacaaacgcatcaaagaactttataaagatggaactcttgaaaaattgtctaaacaattcttcggagacacttatctaccggcagaagctgatattaaactggccgaagcaaccgccaaagaggccaccgccaaggaagccaccgccaaagccaccgcctacttcaaagagggcttagagaagaccatcgccgccaagaaggccgagttagagaaaaccgaggccgacctgaagaaggccgtgaacgagccggagaagcctgcacctgcccctgaaacccctgcaccggaagcacctgcagagcagcctaaacctgcacctgcaccgcaacctgcccctgcacctaaaccggagaaaccggcagagcagccgaagccggagaaaaccgacgaccagcaggccgaggaagactacgcccgtcgtagcgaggaagagtacaaccgcctgacccagcagcaaccgccgaaagccgaaaagccggcccctgcaccgaaaaccggctggaagcaggagaacggcatgtggcaccaccaccaccaccactga

SEQ ID NO: 80 - protein, 424 aa, Artificial Sequence
  Synthetic
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADFQQIMVRLSDGQFDYKIFDKIGVETVIKNQGLDNLKVIELPSDQQPYVYPLLAQGQDELKSFVDKRIKELYKDGTLEKLSKQFFGDTYLPAEADIKLAEATAKEATAKEATAKATAYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWHHHHHH

SEQ ID NO: 81 - DNA, 1671 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaactggccgaggcaaccgccaaagaagccaccgccaaggaagccacagccaaggccaccgcaatggccatggccgacctgaagaaggccgtgaatgagccggagaagccggccgaagaagagccggagaatcctgcaccggcccctaagcctgcaccggcacctcagcctgaaaaaccggcacctgcacctgccccgaagcctgaaaagagcgccgaccagcaggccgaagaggactatgcccgccgcagcgaagaggagtacaaccgtctgacccagcagcagccgccgaaagccgagaaacctgccccggcaccggtgcctaagccggaacagcctgcccctgcaccgaagaccggttggaagcaggaraacggcatgtggcaccaccaccaccaccactga

SEQ ID NO: 82 - protein, 556 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGELAEATAKEATAKEATAKATAMAMADLKKAVNEPEKPAEEEPENPAPAPKPAPAPQPEKPAPAPAPKPEKSADQQAEEDYARRSEEEYNRLTQQQPPKAEKPAPAPVPKPEQPAPAPKTGWKQENGMWHHHHHH

SEQ ID NO: 83 - DNA, 1716 bp, Artificial Sequence
  Synthetic
atgtcatctaaatttatgaagagcgctgcggtgcttggaactgctacacttgctagcttgcttttggtagcttgcggaagcaaaactgctgataagcctgctgattctggttcatctgaagtcaaagaactcactgtatatgtagacgagggatataagagctatattgaagaggttgctaaagcttatgaaaaagaagctggagtaaaagtcactcttaaaactggtgatgctctaggaggtcttgataaactttctcttgacaaccaatctggtaatgtccctgatgttatgatggctccatacgaccgtgtaggtagccttggttctgacggacaactttcagaagtgaaattgagcgatggtgctaaaacagacgacacaactaaatctcttgtaacagctgctaatggtaaagtttacggtgctcctgccgttatcgagtcacttgttatgtactacaacaaagacttggtgaaagatgctccaaaaacatttgctgacttggaaaaccttgctaaagatagcaaatacgcattcgctggtgaagatggtaaaactactgccttcctagctgactggacaaacttctactatacatatggacttcttgccggtaacggtgcttacgtctttggccaaaacggtaaagacgctaaagacatcggtcttgcaaacgacggttctatcgtaggtatcaactacgctaaatcttggtacgaaaaatggcctaaaggtatgcaagatacagaaggtgctggaaacttaatccaaactcaattccaagaaggtaaaacagctgctatcatcgacggaccttggaaagctcaagcctttaaagatgctaaagtaaactacggagttgcaactatcccaactcttccaaatggaaaagaatatgctgcattcggtggtggtaaagcttgggtcattcctcaagccgttaagaaccttgaagcttctcaaaaatttgtagacttccttgttgcaactgaacaacaaaaagtattatatgataagactaacgaaatcccagctaatactgaggctcgttcatacgctgaaggtaaaaacgatgagttgacaacagctgttatcaaacagttcaagaacactcaaccactgccaaacatctctcaaatgtctgcagtttgggatccagcgaaaaatatgctctttgatgctgtaagtggtcaaaaagatgctaaaacagctgctaacgatgctgtaacattgatcaaagaaacaatcaaacaaaaatttggtgaactggccgaagcaaccgccaaagaggccaccgccaaggaagccaccgccaaagccaccgcctacttcaaagagggcttagagaagaccatcgccgccaagaaggccgagttagagaaaaccgaggccgacctgaagaaggccgtgaacgagccggagaagcctgcacctgcccctgaaacccctgcaccggaagcacctgcagagcagcctaaacctgcacctgcaccgcaacctgcccctgcacctaaaccggagaaaccggcagagcagccgaagccggagaaaaccgacgaccagcaggccgaggaagactacgcccgtcgtagcgaggaagagtacaaccgcctgacccagcagcaaccgccgaaagccgaaaagccggcccctgcaccgaaaaccggctggaagcaggagaacggcatgtggcaccaccaccaccaccactga

SEQ ID NO: 84 - protein, 571 aa, Artificial Sequence
  Synthetic
MSSKFMKSAAVLGTATLASLLLVACGSKTADKPADSGSSEVKELTVYVDEGYKSYIEEVAKAYEKEAGVKVTLKTGDALGGLDKLSLDNQSGNVPDVMMAPYDRVGSLGSDGQLSEVKLSDGAKTDDTTKSLVTAANGKVYGAPAVIESLVMYYNKDLVKDAPKTFADLENLAKDSKYAFAGEDGKTTAFLADWTNFYYTYGLLAGNGAYVFGQNGKDAKDIGLANDGSIVGINYAKSWYEKWPKGMQDTEGAGNLIQTQFQEGKTAAIIDGPWKAQAFKDAKVNYGVATIPTLPNGKEYAAFGGGKAWVIPQAVKNLEASQKFVDFLVATEQQKVLYDKTNEIPANTEARSYAEGKNDELTTAVIKQFKNTQPLPNISQMSAVWDPAKNMLFDAVSGQKDAKTAANDAVTLIKETIKQKFGELAEATAKEATAKEATAKATAYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWHHHHHH

SEQ ID NO: 85 - protein, 33 aa, Artificial Sequence
  Synthetic
LIVMFESTAGPCLVIAMFTGIVMSTAGCPAGSC

SEQ ID NO: 86 - protein, 25 aa, Streptococcus pneumoniae
MSSKFMKSAAVLGTATLASLLLVAC

SEQ ID NO: 87 - protein, 19 aa, E. coli
MRYLATLLLSLAVLITAGC

SEQ ID NO: 88 - protein, 6 aa, Artificial Sequence
  Synthetic
HHHHHH

SEQ ID NO: 89 - protein, 10 aa, Artificial Sequence
  Synthetic
MSYYHHHHHH

SEQ ID NO: 90 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 91 - protein, 5 aa, Streptococcus pneumoniae
PAPAP

SEQ ID NO: 92 - protein, 6 aa, Streptococcus pneumoniae
PKEPEQ